SEQUENCE LISTING (84 sequences)

SEQ ID NO: 1 - DNA, 28 bp, Artificial Sequence
  Description of Artificial Sequence Synthetic oligonucleotide
cgacagaaggcgaggacagaagaggaca

SEQ ID NO: 2 - DNA, 28 bp, Artificial Sequence
  Description of Artificial Sequence Synthetic oligonucleotide
ccgttgcgcctgattgatgcccagctcc

SEQ ID NO: 3 - DNA, 28 bp, Artificial Sequence
  Description of Artificial Sequence Synthetic oligonucleotide
gtggcggagactgagagcgcgcgcggcc

SEQ ID NO: 4 - DNA, 28 bp, Artificial Sequence
  Description of Artificial Sequence Synthetic oligonucleotide
cttctgtcgattatcaggtaagcgccgc

SEQ ID NO: 5 - DNA, 28 bp, Artificial Sequence
  Description of Artificial Sequence Synthetic oligonucleotide
ctggtgggtggcggagactgagagcgcg

SEQ ID NO: 6 - DNA, 28 bp, Artificial Sequence
  Description of Artificial Sequence Synthetic oligonucleotide
tggtgctggagctggtgggtggcggaga

SEQ ID NO: 7 - protein, 7 aa, Artificial Sequence
  Description of Artificial Sequence Synthetic peptide
RSDNLAR

SEQ ID NO: 8 - protein, 7 aa, Artificial Sequence
  Description of Artificial Sequence Synthetic peptide
DRSHLAR

SEQ ID NO: 9 - protein, 7 aa, Artificial Sequence
  Description of Artificial Sequence Synthetic peptide
QSGNLAR

SEQ ID NO: 10 - protein, 7 aa, Artificial Sequence
  Description of Artificial Sequence Synthetic peptide
QSNTRIM

SEQ ID NO: 11 - protein, 7 aa, Artificial Sequence
  Description of Artificial Sequence Synthetic peptide
ERGTLAR

SEQ ID NO: 12 - protein, 7 aa, Artificial Sequence
  Description of Artificial Sequence Synthetic peptide
TSANLSR

SEQ ID NO: 13 - protein, 7 aa, Artificial Sequence
  Description of Artificial Sequence Synthetic peptide
TSGNLTR

SEQ ID NO: 14 - protein, 7 aa, Artificial Sequence
  Description of Artificial Sequence Synthetic peptide
HRTSLTD

SEQ ID NO: 15 - protein, 7 aa, Artificial Sequence
  Description of Artificial Sequence Synthetic peptide
RSHSLLR

SEQ ID NO: 16 - protein, 7 aa, Artificial Sequence
  Description of Artificial Sequence Synthetic peptide
HPSARKR

SEQ ID NO: 17 - protein, 7 aa, Artificial Sequence
  Description of Artificial Sequence Synthetic peptide
RSANLTR

SEQ ID NO: 18 - protein, 7 aa, Artificial Sequence
  Description of Artificial Sequence Synthetic peptide
DSSHLEL

SEQ ID NO: 19 - protein, 7 aa, Artificial Sequence
  Description of Artificial Sequence Synthetic peptide
DRSNLTR

SEQ ID NO: 20 - protein, 7 aa, Artificial Sequence
  Description of Artificial Sequence Synthetic peptide
DRSHLTR

SEQ ID NO: 21 - protein, 7 aa, Artificial Sequence
  Description of Artificial Sequence Synthetic peptide
RSDNLSE

SEQ ID NO: 22 - protein, 7 aa, Artificial Sequence
  Description of Artificial Sequence Synthetic peptide
TSSNRKT

SEQ ID NO: 23 - protein, 7 aa, Artificial Sequence
  Description of Artificial Sequence Synthetic peptide
DRSALAR

SEQ ID NO: 24 - protein, 7 aa, Artificial Sequence
  Description of Artificial Sequence Synthetic peptide
RNSDRTK

SEQ ID NO: 25 - protein, 7 aa, Artificial Sequence
  Description of Artificial Sequence Synthetic peptide
RSAHLSR

SEQ ID NO: 26 - protein, 7 aa, Artificial Sequence
  Description of Artificial Sequence Synthetic peptide
TSGHLSR

SEQ ID NO: 27 - protein, 7 aa, Artificial Sequence
  Description of Artificial Sequence Synthetic peptide
LRHHLTR

SEQ ID NO: 28 - protein, 7 aa, Artificial Sequence
  Description of Artificial Sequence Synthetic peptide
RRFTLSK

SEQ ID NO: 29 - protein, 7 aa, Artificial Sequence
  Description of Artificial Sequence Synthetic peptide
RSDVLSE

SEQ ID NO: 30 - protein, 7 aa, Artificial Sequence
  Description of Artificial Sequence Synthetic peptide
KHSTRRV

SEQ ID NO: 31 - protein, 7 aa, Artificial Sequence
  Description of Artificial Sequence Synthetic peptide
RLYTLHK

SEQ ID NO: 32 - DNA, 28 bp, Artificial Sequence
  Description of Artificial Sequence Synthetic oligonucleotide
ctccagaaggggatcatgacctcctcac

SEQ ID NO: 33 - DNA, 28 bp, Artificial Sequence
  Description of Artificial Sequence Synthetic oligonucleotide
aggggcgggcagcgaggcctgggcgggc

SEQ ID NO: 34 - protein, 7 aa, Artificial Sequence
  Description of Artificial Sequence Synthetic peptide
RSADLTR

SEQ ID NO: 35 - protein, 7 aa, Artificial Sequence
  Description of Artificial Sequence Synthetic peptide
QSGDLTR

SEQ ID NO: 36 - protein, 7 aa, Artificial Sequence
  Description of Artificial Sequence Synthetic peptide
RSDHLSE

SEQ ID NO: 37 - protein, 7 aa, Artificial Sequence
  Description of Artificial Sequence Synthetic peptide
LKQHLTR

SEQ ID NO: 38 - protein, 7 aa, Artificial Sequence
  Description of Artificial Sequence Synthetic peptide
DRSHLSR

SEQ ID NO: 39 - protein, 7 aa, Artificial Sequence
  Description of Artificial Sequence Synthetic peptide
DRSNLSR

SEQ ID NO: 40 - protein, 7 aa, Artificial Sequence
  Description of Artificial Sequence Synthetic peptide
LRQNLIM

SEQ ID NO: 41 - protein, 7 aa, Artificial Sequence
  Description of Artificial Sequence Synthetic peptide
TSANLTV

SEQ ID NO: 42 - protein, 7 aa, Artificial Sequence
  Description of Artificial Sequence Synthetic peptide
RSDHLSR

SEQ ID NO: 43 - protein, 7 aa, Artificial Sequence
  Description of Artificial Sequence Synthetic peptide
QRNDRKS

SEQ ID NO: 44 - DNA, 28 bp, Artificial Sequence
  Description of Artificial Sequence Synthetic oligonucleotide
cggcagaaggtgggcggtggcggcggcg

SEQ ID NO: 45 - DNA, 28 bp, Artificial Sequence
  Description of Artificial Sequence Synthetic oligonucleotide
gcggcggcggcagaaggtgggcggtggc

SEQ ID NO: 46 - DNA, 28 bp, Artificial Sequence
  Description of Artificial Sequence Synthetic oligonucleotide
gcggcgctgctgctggtgctggagctgg

SEQ ID NO: 47 - DNA, 28 bp, Artificial Sequence
  Description of Artificial Sequence Synthetic oligonucleotide
tcagagctgcggcgcttacctgataatc

SEQ ID NO: 48 - DNA, 28 bp, Artificial Sequence
  Description of Artificial Sequence Synthetic oligonucleotide
attccgtggcgcacgcgcacccggcctc

SEQ ID NO: 49 - DNA, 28 bp, Artificial Sequence
  Description of Artificial Sequence Synthetic oligonucleotide
cggcggcagaaggtgggcggtggcggcg

SEQ ID NO: 50 - DNA, 28 bp, Artificial Sequence
  Description of Artificial Sequence Synthetic oligonucleotide
ccccaagtgctacaggctccccgagtct

SEQ ID NO: 51 - DNA, 28 bp, Artificial Sequence
  Description of Artificial Sequence Synthetic oligonucleotide
ctgctgctggtgctggagctggtgggtg

SEQ ID NO: 52 - DNA, 28 bp, Artificial Sequence
  Description of Artificial Sequence Synthetic oligonucleotide
cagcaccagcagcagcgccgccgccacc

SEQ ID NO: 53 - DNA, 28 bp, Artificial Sequence
  Description of Artificial Sequence Synthetic oligonucleotide
cgccggcctccagaacgcgctttctcgg

SEQ ID NO: 54 - DNA, 28 bp, Artificial Sequence
  Description of Artificial Sequence Synthetic oligonucleotide
ggtaagcgccgcagctctgaaatccagt

SEQ ID NO: 55 - DNA, 28 bp, Artificial Sequence
  Description of Artificial Sequence Synthetic oligonucleotide
cgggccgcggtgctgaccgccgcgcgca

SEQ ID NO: 56 - DNA, 28 bp, Artificial Sequence
  Description of Artificial Sequence Synthetic oligonucleotide
tacctgataatcgacagaaggcgaggac

SEQ ID NO: 57 - protein, 7 aa, Artificial Sequence
  Description of Artificial Sequence Synthetic peptide
QSSDLSR

SEQ ID NO: 58 - protein, 7 aa, Artificial Sequence
  Description of Artificial Sequence Synthetic peptide
HRSTRNR

SEQ ID NO: 59 - protein, 7 aa, Artificial Sequence
  Description of Artificial Sequence Synthetic peptide
RSDDLTR

SEQ ID NO: 60 - protein, 7 aa, Artificial Sequence
  Description of Artificial Sequence Synthetic peptide
QSTHRNA

SEQ ID NO: 61 - protein, 7 aa, Artificial Sequence
  Description of Artificial Sequence Synthetic peptide
RSADLSR

SEQ ID NO: 62 - protein, 7 aa, Artificial Sequence
  Description of Artificial Sequence Synthetic peptide
DQSNLRA

SEQ ID NO: 63 - protein, 7 aa, Artificial Sequence
  Description of Artificial Sequence Synthetic peptide
RRSDLKR

SEQ ID NO: 64 - protein, 7 aa, Artificial Sequence
  Description of Artificial Sequence Synthetic peptide
RSDSLSR

SEQ ID NO: 65 - protein, 7 aa, Artificial Sequence
  Description of Artificial Sequence Synthetic peptide
DRSDRTK

SEQ ID NO: 66 - protein, 7 aa, Artificial Sequence
  Description of Artificial Sequence Synthetic peptide
YNKDLVK

SEQ ID NO: 67 - protein, 7 aa, Artificial Sequence
  Description of Artificial Sequence Synthetic peptide
LKDTLRR

SEQ ID NO: 68 - protein, 7 aa, Artificial Sequence
  Description of Artificial Sequence Synthetic peptide
DRSTLRQ

SEQ ID NO: 69 - protein, 7 aa, Artificial Sequence
  Description of Artificial Sequence Synthetic peptide
QHGSLSR

SEQ ID NO: 70 - protein, 7 aa, Artificial Sequence
  Description of Artificial Sequence Synthetic peptide
WHSSLHQ

SEQ ID NO: 71 - protein, 7 aa, Artificial Sequence
  Description of Artificial Sequence Synthetic peptide
QSGTRLE

SEQ ID NO: 72 - protein, 7 aa, Artificial Sequence
  Description of Artificial Sequence Synthetic peptide
FRYYLKR

SEQ ID NO: 73 - protein, 7 aa, Artificial Sequence
  Description of Artificial Sequence Synthetic peptide
QRIDLTR

SEQ ID NO: 74 - protein, 7 aa, Artificial Sequence
  Description of Artificial Sequence Synthetic peptide
DRSTRTK

SEQ ID NO: 75 - protein, 7 aa, Artificial Sequence
  Description of Artificial Sequence Synthetic peptide
RRDTLLD

SEQ ID NO: 76 - protein, 7 aa, Artificial Sequence
  Description of Artificial Sequence Synthetic peptide
LAYDRRK

SEQ ID NO: 77 - protein, 7 aa, Artificial Sequence
  Description of Artificial Sequence Synthetic peptide
RSDNLSA

SEQ ID NO: 78 - protein, 7 aa, Artificial Sequence
  Description of Artificial Sequence Synthetic peptide
RNNDRKT

SEQ ID NO: 79 - protein, 7 aa, Artificial Sequence
  Description of Artificial Sequence Synthetic peptide
QSANRTK

SEQ ID NO: 80 - protein, 7 aa, Artificial Sequence
  Description of Artificial Sequence Synthetic peptide
LRQDLKR

SEQ ID NO: 81 - protein, 7 aa, Artificial Sequence
  Description of Artificial Sequence Synthetic peptide
RSDNLSV

SEQ ID NO: 82 - protein, 7 aa, Artificial Sequence
  Description of Artificial Sequence Synthetic peptide
YDYGRYT

SEQ ID NO: 83 - protein, 7 aa, Artificial Sequence
  Description of Artificial Sequence Synthetic peptide
ASWTLTQ

SEQ ID NO: 84 - protein, 7 aa, Artificial Sequence
  Description of Artificial Sequence Synthetic peptide
QNAHRKT

(19) United States
(12) Patent Application Publication
(10) Pub. No.: US 2020/0109406 A1
(43) Pub. Date: Apr. 9, 2020

(54) ENGINEERED GENETIC MODULATORS

(71) Applicant: Sangamo Therapeutics, Inc., Richmond, CA (US)

(72) Inventors: Jeffrey C. Miller, Richmond, CA (US); Bryan Zeitler, Richmond, CA (US)

(21) Appl. No.: 16/591,445

(22) Filed: Oct. 2, 2019

Related U.S. Application Data
(60) Provisional application No. 62/740,156, filed on Oct. 2, 2018.

(51) Int. Cl. C12N 15/62; C07K 14/47; C12N 9/80; C12N 9/10; C12N 9/22

(52) U.S. Cl. CPC C12N 15/62; C07K 14/4702; C07K 2319/81; C12N 9/1029; C12N 9/22; C12N 9/80

(57) ABSTRACT

Genetic modulators comprising two or more artificial transcription factors for use in specific and active modulation of gene expression are provided.

### CROSS-REFERENCE TO RELATED APPLICATIONS

The present application claims the benefit of U.S. Provisional Application No. 62/740,156, filed Oct. 2, 2018, the disclosure of which is hereby incorporated by reference in its entirety.

### TECHNICAL FIELD

The present disclosure is in the field of compositions and methods for modulating gene expression using genetic modulators comprising two or more artificial transcription factors.

### BACKGROUND

Repression or activation of disease-associated genes has been accomplished through the use of engineered transcription factors. Methods of designing and using engineered zinc finger transcription factors (ZFP-TF) are well documented (see for example U.S. Pat. No. 6,534,261), and both transcription activator like effector transcription factors (TALE-TF) and clustered regularly interspaced short palindromic repeat Cas based transcription factors (CRISPR-Cas-TF) have also been described (see review Kabadi and Gersbach (2014) Methods 69(2):188-197). For example, engineered TFs that repress gene expression (repressors) have also been shown to be effective in treating trinucleotide disorders such as Huntingtin's disease (HD) (see, e.g., U.S. Pat. No. 8,956,828 and U.S. Patent Publication No. 2015/0335708) and tauopathies such as Alzheimer's disease (AD) (see, U.S. Publication No. 20180153921).

However, there remains a need for additional methods and compositions that provide enhanced activity and/or specificity for modulation of gene expression.

### SUMMARY

Disclosed herein are genetic modulators comprising two or more artificial transcription factors and methods for making and using these genetic modulators the treatment and/or prevention of diseases. In particular, genetic modulator compositions comprising a plurality of (two or more) artificial transcription factors, in which each artificial transcription factor comprises a DNA-binding domain and functional domain. Surprisingly and unexpectedly, genetic modulators made up of a plurality of artificial transcription factors provide an unexpected synergistic effect in one or more of the following: specificity and/or activity, as compared to compositions comprising a single artificial transcription factor (including at the same dose or at 2× the dose) and/or as compared to any expected additive effect of using multiple artificial TFs. The genetic modulators comprising a plurality of artificial transcription factors modulate gene expression and limit off-target events such that therapeutic effects are achieved, for example repression of mutant Huntingtin (Htt) gene expression for the treatment of Huntington's disease (HD), the repression of a mutant C9orf72 allele for the treatment of amyotrophic lateral sclerosis (ALS), repression of prion protein expression for treatment of prion disease; repression of α-synuclein for treatment of synucleinopathies such as Parkinson's disease (PD) and/or dementia with Lewy bodies (DLB) and/or repression of MAPT gene expression for the treatment of tauopathies such as AD, FTD, PSP, CBD and/or seizures. Thus, provided herein are methods and compositions for modulating gene expression in vitro, ex vivo and in vivo.

In one aspect, described herein are genetic modulators comprising two or more (a plurality of) artificial transcription factors in which the genetic modulators modulate gene expression (activate or repress) at higher levels (from between about 1 to 10 or more-fold more) as compared to gene expression levels when each individual artificial transcription factor is administered separately. The genetic modulators thus exhibit synergistic effects as compared to individual transcription factors and as compared to expected (e.g. additive) levels of gene modulation using combinations of transcription factors. In certain embodiments, the genetic modulators comprise 2, 3, 4, 5, or more artificial transcription factors, each artificial transcription factor comprising (i) any DNA-binding domain (e.g., zinc finger protein (ZFP), TAL-effector domain, sgRNA of CRISPR/Cas system, etc.) that binds to a target site of 12 or more (e.g., 12, 13, 14, 15, 16, 17, 18, 19, 20, 21, 22, 23, 24, 25 or more) nucleotides and (ii) a functional domain (e.g., a transcriptional activation domain, a transcriptional repression domain, a domain from a DNMT protein, a histone deacetylase etc.,) such that the genetic modulator modulates gene expression.

The DNA-binding domain of the artificial transcription factors as described herein may bind to any target site of at least 12 nucleotides (contiguous or non-contiguous) in any selected target gene. Furthermore, the DNA-binding domains of the artificial transcription factors may bind to the same, different or overlapping target sites. In certain embodiments, the DNA-binding domains bind to different, non-overlapping targets. Alternatively, in some embodiments, at least two of the DNA-binding domains bind to overlapping target sites. In other embodiments, the DNA-binding domains bind to target sites within about 800 base pairs of each other. In other embodiments, the DNA-binding domains bind to target sites within about 10,000 (or more) base pairs of each other. In still further embodiments, the DNA-binding domains bind near (e.g., within 0 to about 600 base pairs (or any value therebetween)) on either side of the transcription start site (TSS), including 0- about 300 base pairs (or any value therebetween), 0- about 200 (or any value therebetween), or 0- about 100 base pairs (or any value therebetween) of the target gene to be modulated. Some or all of the DNA-binding domains of the artificial transcription factors bind to the sense strand in a double stranded target (e.g., endogenous gene); some or all may bind to the antisense strand; or one or more may bind to the sense strand and one or more may bind to the antisense strand.

The compositions as described herein may target any gene for modulation (e.g., repression). In certain embodiments, the target gene is a tau (MAPT) gene or a Htt gene. In some embodiments, the target is a mutant C9orf72 gene. In other non-limiting embodiments, the target gene is an SNCA gene, an SMA gene, an ATXN1 gene, an ATXN2 gene, an ATXN3 gene, an ATXN7 gene, a PRNP gene, an Ube3a-ATS-encoding gene, a DUX4 gene, a PGRN gene, an MECP2 gene, an FMR1 gene, a CDKL5 gene, a LRKK2 gene, an APOE gene, a RHO gene, or any gene wherein a modulation of gene expression is desired. Any combination of DNA-binding domains can be used in the genetic modulators described herein (e.g., any combination of ZFPs, TALEs and/or sgRNAs, overlapping and/or non-overlapping target sites, proximity to the TSS, sense or antisense strand bound, etc.).

In certain embodiments, one or more of the DNA-binding domains of the artificial transcription factors of the genetic modulator comprise a ZFP to form a ZFP-TF. Any of the zinc finger proteins described herein may include 1, 2, 3, 4, 5, 6 or more zinc fingers, each zinc finger having a recognition helix that binds to a target subsite in the selected target sequence(s) (e.g., gene(s)). The target subsites may be contiguous or non-contiguous. In certain embodiments, the genetic modulator comprises a plurality of ZFP-TFs, for example a plurality of ZFP-TF repressors. The ZFPs may bind to any target sites in the selected gene.

In other embodiments, one or more of the DNA-binding domains of the artificial transcription factors of the genetic modulator comprise a TAL-effector domain protein (TALE), to form a TALE-TF in which the repeat variable diresidue (RVD) regions bind to the selected target site of 12 or more nucleotides. In some embodiments, at least one RVD has non-specific DNA binding characteristics. In still other embodiments, one or more of the DNA-binding domains of the artificial transcription factors of the genetic modulators described herein comprise a single guide RNA (to form a CRISPR/Cas-TF system) that binds to the selected target sequence. The DNA-binding domains may be all of the same type or may include artificial transcription factors with different DNA-binding domains. Thus, the two or more artificial transcription factors of the genetic modulators described herein may be of the same type (e.g., all ZFP-TFs, all TAL-TFs, all CRISPR/Cas-TFs) or may include a combination of different types of artificial transcription factors (e.g., ZFP-TFs, TALE-TFs, CRISPR/Cas-TFs, etc.).

The artificial transcription factors described herein (ZFP-TFs, TALE-TFs, CRISPR/Cas-TFs, etc.) can comprise one or more functional domains placed in operative linkage with the DNA-binding domain. The functional domain can comprise, for example, a transcriptional activation domain or a transcriptional repression domain. By selecting either an activation domain or repression domain for use with the DNA-binding domain, such molecules can be used either to activate or to repress expression of the target gene. In any of the artificial TFs of the genetic modulators described herein, the functional domain (e.g., transcriptional activation domain or repression domain) may be a wild-type (e.g., P65, KRAB, KOX). In certain embodiments, the functional domain comprises a codon-diversified repression domain to prevent recombination between ZFPs linked in cis (e.g., nKOX, mKOX, cKOX). The artificial TFs of the genetic modulators may include the same or different functional domains (e.g., different combinations of wild-type and or modified (e.g. codon-diversified) repression domains). In certain embodiments, the functional or regulatory domains can play a role in histone post-translational modifications. In some instances, the functional domain is a histone acetyltransferase (HAT), a histone deacetylase (HDAC), a histone methylase, or an enzyme that sumolyates or biotinylates a histone or other enzyme domain that allows post-translation histone modification regulated gene repression (Kousarides (2007) Cell 128:693-705). In other embodiments, the artificial transcription factor comprises a DNMT domain (e.g., DNMT1, DNMT3A, DNMT3B, DNMT3L).

In some embodiments, the methods and compositions of the invention are useful for treating eukaryotes. In certain embodiments, the activity of the functional (regulatory) domain is regulated by an exogenous small molecule or ligand such that interaction with the cell's transcription machinery will not take place in the absence of the exogenous ligand. Such external ligands control the degree of interaction of the ZFP-TF, CRISPR/Cas-TF or TALE-TF with the transcription machinery. The regulatory domain(s) may be operatively linked to any portion(s) of one or more of the ZFPs, sgRNA/dCas or TALEs, including between one or more ZFPs, sgRNA/dCas or TALEs, exterior to one or more ZFPs, sgRNA/dCas or TALEs and any combination thereof. In preferred embodiments, the regulatory domain results in a repression of gene expression of the targeted gene.

In certain embodiments, the genetic modulators comprising two or more artificial transcription factors are repressors and repress expression of the target gene by at least 50% to 100% (or any value therebetween) as compared to wild-type expression levels. In some embodiments, the genetic repressors repress expression of the target gene by at least 75% as compared to wild-type expression levels. In still further embodiments, the genetic modulators are repressors and repress expression by at least 10% to 100% as compared to expression levels when the gene is modulated by a single genetic modulator (artificial transcription factor). In other embodiments, the genetic modulators are activators and activate gene expression by between about 1 to 5-fold or more (including up to 100-fold or more) as compared to wild-type expression levels and/or expression levels when the gene is modulated by a single genetic modulator (see Perez-Pinera et al (2013) Nat Method 10(3):239-42). Any of the genetic modulators described herein may further reduce off-target gene modulation (e.g., more than about 50% or about 75% or about 90% or about 100% of off-target modulation).

The genetic modulators described herein may be provided to the subject in any form, including in polynucleotide and/or protein form as well provided as pharmaceutical compositions comprising such polynucleotides and/or proteins.

In some aspects, the genetic modulators (or a component thereof, for example one or more DNA-binding domains of the artificial transcription factors) are provided in polynucleotide form using one or more polynucleotides. In certain embodiments, a single polynucleotide is used to deliver all the artificial transcription factors of the genetic modulator, while in other embodiments, two or more polynucleotides (of the same or different types) are used to deliver the plurality of artificial transcription factors in any combination or order. In certain embodiments, the polynucleotide is a gene delivery vector comprising any of the polynucleotides (e.g., encoding the genetic modulators (repressors)) as described herein. In certain embodiments, the vector is an adenovirus vector (e.g., an Ad5/F35 vector), a lentiviral vector (LV) including integration competent or integration-defective lentiviral vectors, or an adenovirus associated viral vector (AAV). In certain embodiments, the genetic modulator(s) are carried on at least one AAV vector (or pseudotype or variant thereof), including but not limited to one or more AAV1, AAV2, AAV3, AAV4, AAVS, AAV6, AAV8, AAV 8.2, AAV9, AAV rh10, pseudotypes of these vectors (e.g., as AAV2/8, AAV2/5, AAV2/6, AAV2/9, etc.), including, but not limited to, AAV vector variants known in the art (e.g. U.S. Pat. Nos. 9,585,971 and 7,198,951; U.S. Publication No. 20170119906). In some embodiments, the AAV vector is an AAV variant capable of crossing the blood-brain barrier (e.g. U.S. Pat. No. 9,585,971). In some embodiments, the artificial transcription factors are carried by one or more multi-cistronic polynucleotides (e.g., AAV vector or mRNA), namely a polynucleotide that encodes at least two or more of the artificial transcription factors of the genetic modulators described herein. In some embodiments, a single multi-cistronic polynucleotide (e.g., AAV vector or mRNA) encodes all the artificial transcription factors of the genetic modulator described herein. In multi-cistronic polynucleotides the coding sequences may be separated by self-cleaving peptides or IRES sequences.

In certain embodiments, the two or more artificial transcription factors of the genetic modulators described herein are encoded by one or more vectors, including viral and non-viral gene delivery vehicles (e.g., as mRNA, plasmids, AAV vectors, lentiviral vectors, Ad vectors) encoding the genetic modulators as described herein. In some embodiments, the two or more artificial transcription factors of the genetic modulators described herein are encoded by separate vectors. In some embodiments, the components (e.g. sgRNA) of the two or more artificial transcription factors of the genetic modulators described herein are encoded separately from other components (e.g. Cas). In certain embodiments, the polynucleotide is an mRNA. In some aspects, the mRNA may be chemically modified (See e.g. Kormann et al., (2011) Nature Biotechnology 29(2):154-157). In other aspects, the mRNA may comprise a cap (e.g. an ARCA cap (see U.S. Pat. Nos. 7,074,596 and 8,153,773)). In further embodiments, the mRNA may comprise a mixture of unmodified and modified nucleotides (see U.S. Patent Publication No. 2012/0195936). In still further embodiments, the mRNA may be multi-cistronic, e.g., include two or more transcription factors linked by sequence such as an IRES or a self-cleaving peptide.

The invention also provides methods and uses for modulating (e.g., repressing) gene expression in a subject in need thereof, including by providing to the subject one or more polynucleotides, one or more gene delivery vehicles, and/or a pharmaceutical composition comprising genetic modulators as described herein. In certain embodiments, the compositions described herein are used to repress gene expression in the subject, including for treatment and/or prevention of a disease associated with aberrant expression of the gene (e.g., tau in a tauopathy, mutant C9orf72 for the treatment of ALS, mutant Htt in HD; prion genes for treatment of prion disorders; α-synuclein for treatment of PD and/or other genes as described above). Thus, in certain embodiments, the compositions described herein are used to repress tau expression in the subject, including for treatment and/or prevention of AD while in other embodiments, the compositions described herein are used to repress Htt expression in the subject, including for treatment and/or prevention of HD (e.g., by reducing the amount of mutant Htt in the subject). In certain embodiments, the compositions described herein are used to repress mutant C9Orf72 (e.g. expanded) expression in a subject, including for the treatment and/or prevention of ALS. In certain embodiments, the compositions described herein are used to repress prion expression in a subject, including for the treatment and/or prevention of prion diseases. In still further embodiments, the compositions described herein are used to repress α-synuclein expression in a subject, including for the treatment and/or prevention of PD.

The compositions described herein reduce gene expression levels for sustained periods of time (e.g., about 4 weeks, about 3 months, about 6 months to about a year or more) and may be used in any part of the subject. In certain embodiments, the compositions are used in the brain (including but not limited to the frontal cortical lobe including, e.g. the prefrontal cortex, parietal cortical lobe, occipital cortical lobe; temporal cortical lobe including e.g. the entorhinal cortex, hippocampus, brain stem, striatum, thalamus, midbrain, cerebellum) and spinal cord (including but not limited to lumbar, thoracic and cervical regions).

The compositions described herein may be provided to the subject by any administration means, including but not limited to, intravenous, intramuscular, intracerebroventricular, intrathecal, intracranial, intravenous, orbital (retro-orbital (RO)) and/or intracisternal administration. Delivery may be to any part of a subject, including intravenously, intramuscularly, orally, mucosally, etc. In certain embodiments, delivery is to any brain region, for example, the hippocampus or entorhinal cortex by any suitable means including via the use of a cannula or any other delivery technology. Any AAV vector that provides widespread delivery of the repressor to brain of the subject, including via anterograde and retrograde axonal transport to brain regions not directly administered the vector (e.g., delivery to the putamen results in delivery to other structures such as the cortex, substantia nigra, thalamus, etc.). In certain embodiments, the subject is a human and in other embodiments, the subject is a non-human primate or a rodent. The administration may be in a single dose, in multiple administrations given at the same time or in multiple administrations (at any timing between administrations).

Furthermore, in any of the methods described herein, the genetic modulators can be delivered at any concentration (dose) that provides the desired effect. In preferred embodiments, the genetic modulator is delivered using an adeno-associated virus (AAV) vector at about 10,000 to about 500,000 vector genomes/cell (or any value therebetween). In some embodiments, the genetic modulator-AAV is delivered at a dose of about 10,000 to about 100,000, or from about 100,000 to about 250,000, or from about 250,000 to about 500,000 vector genomes (VG)/cell (or any value therebetween). In certain embodiments, the repressor is delivered using a lentiviral vector at a multiplicity of infection (MOI) of between about 250 and about 1,000 (or any value therebetween). In other embodiments, the genetic modulator is delivered using a plasmid vector at about 0.01- about 1,000 ng/about 100,000 cells (or any value therebetween). In some embodiments, the genetic modulator is delivered using a plasmid vector from about 0.01 to about 1, from about 1 to about 100, from about 100 to about 500, or from about 500 to about 1000 ng/about 100,000 cells (or any value therebetween). In other embodiments, the genetic modulator is delivered as mRNA at about 0.01 to about 3000 ng/about 100,000 cells (or any value therebetween). In other embodiments, the genetic modulator is delivered using an adeno-associated virus (AAV) vector at a fixed volume of about 1-300 μL to the brain parenchyma at between about 1E11-1E14 VG/mL. In other embodiments, the repressor is delivered using an adeno-associated virus (AAV) vector at a fixed volume of between about 0.1-25 mL to the CSF at between about 1E11-1E14 VG/mL.

In another aspect, provided herein are methods of making compositions comprising two or more (synergistic) artificial transcription factors (TFs). In certain embodiments, the methods involve screening a plurality of artificial transcription factors (e.g., ZFP-TFs) targeted to a selected gene for their effect, individually and in combinations, on gene expression; and identifying synergistic combinations of the artificial ZFP-TFs. Screening is conducted using known techniques. See, also, Examples. In certain embodiments, the methods involve the step of selecting (i) two or more artificial transcription factors that bind to target sites that are about 1-600 (or any value therebetween) base pairs apart and/or (ii) selecting two or more artificial transcription factors in which the functional domains of the TFs, when bound to the target gene, are about 1-600 (or any value therebetween) base pairs apart from each other. In certain embodiments, the methods comprise screening for synergistic artificial TFs that bind to target sites in target sequence a periodic manner, for example, target sites separated by spacings spanning approximately 80-100 nucleotides (or any value therebetween) in the target site, including but not limited to target sites separated by approximately 80 base pairs (e.g., target sites separated by between about 0-80 base pairs; about 160 to 240 base pairs; about 320 to 400 base pairs or between about 480 to 560 base pairs) and/or target sites separated by approximately 100 base pairs (e.g., target sites separated by between about 0 to about 100 base pairs; about 200 to about 300 base pairs; or between about 400 to about 500 base pairs). In certain embodiments, the target sites are separated by 0 to about 80 (or any value therebetween); 0 to about 100 (or any value therebetween); about 160 to 240 (or any value therebetween); about 200 to about 300 (or any value therebetween); about 220 to about 300 (or any value therebetween); about 300 to approximately 0 to about 80 (or any value therebetween), approximately 160 to about 220 (or any value therebetween), approximately 260 to about 400 (or any value therebetween), or approximately 500 to about 600 (or any value therebetween) base pairs apart.

In certain aspects, any of the methods described herein comprise screening for synergistic artificial TFs whose functional domains are separated from each other in a periodic manner, for example, functional domains separated by spacings spanning approximately 80-100 nucleotides (or any value therebetween) in the target gene, including but not limited to synergistic TFs in which the functional domains are separated by approximately 80 base pairs (e.g., functional domains separated by between about 0 to about 80 base pairs; about 160 to about 240 base pairs; about 320 to about 400 base pairs or about 480 to about 560 base pairs) and/or functional domains separated by approximately 100 base pairs target sites separated by between about 0 to about 100 base pairs; about 200 to about 300 base pairs; or between about 400 to about 500 base pairs). In certain embodiments, the functional domains that are approximately 0 to 80 (or any value therebetween), approximately 160 to 220 (or any value therebetween), approximately 260 to 400 (or any value therebetween), or approximately 500 to 600 (or any value therebetween) base pairs apart from each other. In still further embodiments, the methods comprise screening for synergistic artificial TFs that bind to target sites that are within about 800 base pairs (or any value therebetween) on either side of the transcription start site (TSS), preferably within about 600 base pairs on either side of the TSS, even more preferably within about 300 base pairs of the TSS. In certain embodiments, the TFs bind to target sites that are between the TSS and +200 (or any value therebetween) of the TSS. The methods may further comprise screening for synergistic TFs that bind to the same antisense (−) or sense (+) strand or to different strands (+/− in either orientation). The methods of the invention identify artificial TFs exhibit synergistic effects (an increase in activity and/or specificity) of more than about 1-fold, about 2-fold, about 3-fold, about 4-fold, about 5-fold, about 6-fold, about 7,-fold, about 8-fold or more as compared to individual TFs (and/or expected additive effects).

Thus, provided herein are methods for treating and/or preventing a disorder associated with undesirable expression of one or more genes using the methods and compositions described herein. In some embodiments, the methods involve compositions where the polynucleotides and/or proteins (or pharmaceutical compositions comprising the polynucleotides and/or proteins) may be delivered using a viral vector, a non-viral vector (e.g., plasmid) and/or combinations thereof. Administration of compositions as described herein (proteins, polynucleotides, cells and/or pharmaceutical compositions comprising these proteins, polynucleotides and/or cells) result in a therapeutic (clinical) effect, including, but not limited to, amelioration or elimination of any the clinical symptoms associated with the disorders (e.g., HD, AD, ALS, other tauopathies or seizure) as well as an increase in function and/or number of CNS cells (e.g., neurons, astrocytes, myelin, etc.). In certain embodiments, the compositions and methods described herein reduce gene expression (as compared to controls not receiving the genetic modulators as described herein) by at least about 30%, or about 40%, preferably by at least about 50%, even more preferably by at least about 70%, or by at least about 80%, or by about 90%, or by greater than 90%. In some embodiments, at least about 50% reduction is achieved. Use of any of the compositions in the methods described herein, the methods can yield about 50% or greater, about 55% or greater, about 60% or greater, about 65% or greater, about 70% or greater, about 75% or greater, about 85% or greater, about 90% or greater, about 92% or greater, or about 95% or greater repression of the target alleles (e.g., Htt, prion, SNCA, tau or C9ORF72) in one or more cells (e.g., HD, ALS or AD neurons) of the subject.

Thus, in other aspects, described herein is a method of preventing and/or treating a disease associated with undesirable gene expression (e.g., HD, AD, ALS) in a subject, the method comprising administering a modulator of an allele to the subject using one or more AAV vectors. In certain embodiments, the AAV encodes a genetic modulator and is administered to the CNS (brain and/or CSF) via any delivery method including but not limited to, intracerebroventricular, intrathecal, or intracisternal delivery. In other embodiments, the AAV encoding the genetic modulator is administered directly into the parenchyma (e.g., hippocampus and/or entorhinal cortex) of the subject. In other embodiments, the AAV encoding the genetic modulator is administered intravenously (IV). In any of the methods described herein, the administering may be done once (single administration), by multiple administrations at the same time, or may be done multiple times (with any time between administrations) at the same or different doses per administration. When administered multiple times, the same or different dosages and/or delivery vehicles of modes of administration may be used (e.g., different AAV vectors administered IV and/or ICV). In some embodiments, the methods include methods of reducing the aggregation of mutant proteins in the subject (e.g., reducing neurofibrillary tangles (NFTs) characteristic of tau aggregation; reducing mutant Htt aggregation; reducing the aggregates of proteins derived from incomplete RNA transcripts of expanded GGGGCC in the C9ORF72 gene ALS) for example in AD neurons of a subject with AD, or HD neurons of a subject with HD, or ALS neurons of a subject with ALS; methods of reducing apoptosis in a neuron or population of neurons (e.g., an HD or AD neuron or population of HD or AD neurons); methods of reducing nuclear foci comprising incomplete RNA transcripts of the expanded GGGGCC locus in ALS neurons; methods of reducing neuronal hyperexcitability; methods of reducing amyloid beta induced toxicity (e.g. synapse loss and/or neuritic dystrophy); and/or methods of reduce loss to one or more cognitive functions in HD or AD subjects, all in comparison with a subject not receiving the method, or in comparison to the subject themselves prior to receiving the methods. Thus, the methods described herein result in reduction in biomarkers and/or symptoms of HD or tauopathies, including one or more the following: neurotoxicity, gliosis, dystrophic neurites, spine loss, excitotoxicity, cortical and hippocampal shrinkage, dendritic tau accumulation, cognitive (e.g., the radial arm maze and the Morris water maze in rodent models, fear conditioning, etc.), and/or motor deficits.

In some aspects, the methods and compositions of the invention for reducing the amount of a pathogenic species (e.g., tau, Htt, C9ORF72, prion, SNCA encoded protein) in a cell are provided. In some embodiments, the methods result in a reduction of hyperphosphorylated tau. In some instances, the reduction of hyperphosphorylated tau results in a reduction of soluble or granular tau. In other embodiments, the reduction of pathogenic tau species decreases tau aggregation and causes a reduction in neurofibrillary tangles (NFTs) as compared to a cell or subject that has not been treated following the methods and/or with the compositions of the invention. In further embodiments, the methods of reversing the amount of NFTs observed in a cell are provided. In still further embodiments, the methods and compositions of the invention cause a slowing of the propagation of pathogenic tau species (NFTs, hyperphosphorylated tau) within the brain of a subject. In some embodiments, propagation of pathogenic tau across the brain is halted, and in other embodiments, propagation of pathogenic tau across the brain is reversed. In further embodiments, the number of dystrophic neurites associated with amyloid β plaques in the brain is reduced. In some embodiments, the number of dystrophic neurites is reduced to the levels found in an age-matched wild type brain. In further embodiments, provided herein are methods and compositions for reducing hyperphosphorylated tau associated with amyloid β plaques in the brain of a subject. In still further embodiments, the compositions (Htt repressors) and methods described herein provide a therapeutic benefit in HD subjects, for example by reducing cell death, decreasing apoptosis, increasing cellular function (metabolism) and/or reducing motor deficiency in the subjects. In some embodiments, provided herein are methods and compositions for reducing the consequences associated with mutant C9ORF72 expansion. The pathology associated with this expansion (from approximately 30 copies in the wild type human genome to hundreds or even thousands in fALS patients) appears to be related to the formation of unusual structures in the DNA and to some type of RNA-mediated toxicity (Taylor (2014) Nature 507:175). Incomplete RNA transcripts of the expanded GGGGCC form nuclear foci in fALS patient cells and also the RNAs can also undergo repeat-associate non-ATP—dependent translation, resulting in the production of three proteins that are prone to aggregation (Gendron et al (2013) Acta Neuropathol 126:829). In some embodiments, provided herein are methods and compositions for reducing the consequences associated with aggregation of α-synuclein. The pathology associated with this aggregation appears to be related to the misfolding and aggregation of alpha-synuclein in synucleinopathies such as PD and dementia with Lewy bodies (DLB). In other embodiments are methods and compositions for reducing the consequences associated with formation of mutant prion strains.

In some embodiments, following administration to the subject, the sequences encoding two or more of the artificial transcription factors of the genetic modulators (e.g., genetic repressors) as described herein (e.g., ZFP-TF, TALE-TF or CRISPR/Cas-TF) are inserted (integrated) into the genome while in other embodiments the sequences encoding two or more of the artificial transcription factors of the genetic modulator are maintained episomally. Alternatively, sequences encoding one or more of the artificial transcription factors may integrated into the genome and the sequences encoding the remaining one or more artificial transcription factors may be maintained episomally. In some instances, the nucleic acid encoding the TF fusion is inserted (e.g., via nuclease-mediated integration) at a safe harbor site comprising a promoter such that the endogenous promoter drives expression. In other embodiments, the repressor (TF) donor sequence is inserted (via nuclease-mediated integration) into a safe harbor site and the donor sequence comprises a promoter that drives expression of the repressor. In some embodiments, the sequence encoding the genetic modulator is maintained extrachromosomally (episomally) after delivery, and may include a heterologous promoter. The promoter may be a constitutive or inducible promoter. In some embodiments, the promoter sequence is broadly expressed while in other embodiments, the promoter is tissue or cell/type specific. In preferred embodiments, the promoter sequence is specific for neuronal cells. In other preferred embodiments, the promoter chosen is characterized in that it has low expression. Non-limiting examples of preferred promoters include the neural specific promoters NSE, CMV, Synapsin, CAMKiia and MECPs. Non-limiting examples of ubiquitous promoters include CAS and Ubc. Further embodiments include the use of self-regulating promoters as described in U.S. Patent Publication No. 20150267205.

Kits comprising one or more of the compositions (e.g., genetic modulators, polynucleotides, pharmaceutical compositions and/or cells) as described herein as well as instructions for use of these compositions are also provided. The kits comprise one or more of the genetic modulators (e.g., repressors) and/or polynucleotides comprising components of and/or encoding the modulators (or components thereof) as described herein. The kits may further comprise cells (e.g., neurons), reagents (e.g., for detecting and/or quantifying the protein encoded by the target gene, for example in CSF) and/or instructions for use, including the methods as described herein.

Thus, described herein are compositions comprising two or more artificial transcription factors (TFs), each artificial transcription factor comprises a DNA-binding domain and functional domain (e.g., a transcriptional activation domain, a transcriptional repression domain, a domain from a DNMT protein such as DNMT1, DNMT3A, DNMT3B, DNMT3L, a histone deacetylase (HDAC), a histone acetyltransferase (HAT), a histone methylase, or an enzyme that sumolyates or biotinylates a histone and/or other enzyme domain that allows post-translation histone modification regulated gene repression), wherein the artificial transcription factors synergistically modulate (activate or repressor) gene expression in a cell. The target gene may be tau (MAPT) gene, a Htt gene, a mutant Htt gene, a mutant C9orf72 gene, a SNCA gene, a SMA gene, an ATXN2 gene, an ATXN3 gene, a PRP gene, an Ube3a-ATS encoding gene, a DUX4 gene, an PGRN gene, a MECP2 gene, an FMR1 gene, a CDKLS gene, and/or a LRKK2. The cell may be isolated or in a living subject. The synergistic TF compositions described herein can exhibit 1-, 2-, 3-, 4-, 5-, 6-, 7-, 8-fold or more modulation of the target gene as compared to wild-type expression levels (and/or untreated controls). The DNA-binding domain may bind to a target site of 12 or more nucleotides and may be a zinc finger protein (ZFP), TAL-effector domain, and/or a sgRNA of CRISPR/Cas system. The two or more artificial transcription factors of the composition may: (i) bind to any target site of at least 12 nucleotides in a selected target gene; (ii) bind to target sites within 10,000 or more base pairs of each other; (iii) bind to target sites within 0 to 300 base pairs on either side of the transcription start site (TSS) of the target gene to be modulated; and/or (iv) bind to the sense and/or anti-sense strand in a double stranded target. Gene modulation (e.g., repression) may by at least 50% to 100% as compared to wild-type expression levels. The activity of the functional domain may be regulated by an exogenous small molecule or ligand such that interaction with the cell's transcription machinery will not take place in the absence of the exogenous ligand. Also described herein are pharmaceutical compositions comprising one or more synergistic TF compositions.

Cells (e.g., isolated or in a living subject) comprising one or more compositions and/or polynucleotides encoding the synergistic TFs of the one or more compositions are also provided. Cells can include neurons, glial cells, ependymal cells, hepatocytes, neuroepithelial cells, optionally an HD or AD neuron or glial cell, or hepatocyte. The polynucleotides encoding the synergistic TFs may be stably integrated into the genome of the cell and/or may be maintained episomally. The compositions can reduce gene expression by at 30%, 40%, 50% or more as compared to controls not receiving the genetic modulators or as compared to cells or subjects receiving a single TF of the synergistic compositions.

Methods of modulating gene expression in a subject (e.g., in a neuron of the subject) with a central nervous system (CNS) disease or disorder are also provided, the method comprising: administering one or more compositions described herein to a subject in need thereof. The CNS disease or disorder may be Huntington's Disease (HD) (by repression of Htt), Amyotrophic lateral sclerosis (ALS) (by repression of a C9orf gene), a prion disease (by repression of a prion gene), Parkinson's Disease (PD) (by repression of α-synuclein expression), dementia with Lewy bodies (DLB) (by repression of α-synuclein expression) and/or a tauopathy (by repression of MAPT), optionally wherein biomarkers, pathogenic species and/or symptoms of the CNS disease or disorder are reduced by the gene modulation (e.g., neurotoxicity, gliosis, dystrophic neurites, spine loss, excitotoxicity, cortical and hippocampal shrinkage, dendritic tau accumulation, cognitive deficits, motor deficits, dystrophic neurites associated with amyloid β plaques, tau pathogenic species, mHtt aggregates, hyperphosphorylated tau, soluble tau, granular tau, tau aggregation, and/or neurofibrillary tangles (NFTs) are reduced). The composition comprising the synergistic artificial transcription factors may be provided (to a cell or subject) using one or more polynucleotides (e.g., non-viral or viral vectors). Non-viral vectors include plasmid and/or single or multi-cistronic mRNA vectors. Viral vectors that may be used for delivery of the one or more compositions include one or more of: adenovirus vectors, lentiviral vectors (LV) and/or adenovirus associated viral vectors (AAV). In any of these methods, gene expression may be reduced for a period of 4 weeks, 3 months, 6 months to year or more in the brain of subject. Further, intravenous, intramuscular, intracerebroventricular, intrathecal, intracranial, mucosal, oral, intravenous, orbital and/or intracisternal administration may be used, including but not limited to the frontal cortical lobe, the parietal cortical lobe, the occipital cortical lobe; the temporal cortical lobe, the hippocampus, the brain stem, the striatum, the thalamus, the midbrain, the cerebellum and/or to the spinal cord of the subject. The composition may be delivered using: (i) an adeno-associated virus (AAV) vector at 10,000-500,000 vector genome/cell; (ii) a lentiviral vector at MOI between 250 and 1,000; (iii) a plasmid vector at 0.01-1,000 ng/100,000 cells; and/or (iv) mRNA (single mRNAs or multi-cistronic) at 0.01-3000 ng/100,000 cells. The methods may involve delivering an AAV vector (carrying the synergistic TF compositions) at a dose of 10,000 to 100,000, or from 100,000 to 250,000, or from 250,000 to 500,000 vector genomes (VG)/cell; at a fixed volume of 1-300 μL to the brain parenchyma at 1E11-1E14 VG/mL and/or at a fixed volume of 0.5-10 mL to the CSF at 1E11-1E14 VG/mL.

Methods of making a composition comprising synergistic artificial transcription factors as described herein are also provide, the methods comprising: screening individual and combinations of two or more artificial transcription factors targeted to a selected gene for their effect on gene expression; and identifying synergistic combinations of the artificial ZFP-TFs. The two or more artificial transcription factors screened may: (i) bind to target sites and/or comprise functional domains that are 1-600 base pairs apart; (ii) bind to target sites that are approximately 1 to 80; 160 to 220; 260 to 400; or 500 to 600 base pairs apart; (iii) comprise functional domains that are separated from each other by approximately 1 to 80; 260 to 400; or 500 to 600 base pairs apart; (iv) bind to target sites that are within 400 base pairs on either side of the transcription start site (TSS); and/or (v) bind to the same antisense (−) or sense (+) strand or to different strands in either orientation). Synergistic artificial TFs obtained by these methods may be at least 2-fold more active than the individual TFs.

### BRIEF DESCRIPTION OF THE DRAWINGS

FIG. 1 depicts exemplary results of repression of the tau (MAPT) gene by the indicated ZFP-TF in Neuro2A cells transfected with mRNA encoding the artificial transcription factors (e.g. ZFP repressors). The top two panels (“ZFP 1” and ZFP 2″) show results of the indicated ZFP-TF (see, also, U.S. Publication No. 20180153921) when used alone. The 3^{rd} panel (“ZFP 1+2”) shows results when two of the indicated ZFPs are used together at the equivalent dose of the individual transfections. Controls are shown at right of the third panel (ZFP 52288 as a positive control which also targets MAPT (see Table 1) (“288”); a negative control ZFP that targets BCL11A (“BCL”); GFP (“GFP”); and a mock transfected control (“MCK”)). The top three panels show tau repression at 3 different dosages (30, 10 and 3 ng mRNA from left to right) for each ZFP-TF. The shaded graphs are also shown in expanded form beneath the shaded areas (ZFP 52322, ZFP 52335, and ZFP 52322 and 52335 shown on the left and ZFP 52364, ZFP 52374 and ZFP 52364 and 52374 shown on the right). Tau repression at 6 different doses (300, 100, 30, 10, 3 and 1 ng mRNA from left to right) of ZFP-TF are shown.

FIG. 2 are graphs depicting surprising synergistic effects of using two or more ZFP-TF repressors and the method used to derive a synergy score. The left panel shows normalized tau expression in Neuro2A cells following transient mRNA transfection at the indicated levels of the ZFP repressors. The middle panel shows the interpolated level of expected normalized tau repression (blue line, inverted triangles) for the stronger single repressor 52322 if it were transfected at 2× the dose, in order to account for the potential effect of the additional amount of mRNA transfected in the combined reaction. The right panel shows the unexpected synergy and its score calculated as the ratio of the expected repression (inverted triangles) and observed repression when the ZFP combination is used (circles).

FIG. 3 depicts exemplary results of repression of the tau (MAPT) gene by the indicated ZFP-TF in Neuro2A cells transfected with mRNA encoding the ZFP repressors and the corresponding synergy score for each combination. The top two panels (“ZFP 1” and ZFP 2”) show results of the indicated ZFP-TF (see, also, U.S. Publication No. 20180153921) when used alone. Controls are shown at right of the third panel (ZFP 52288 as a positive control which also targets MAPT (see Table 1) (“288”); a negative control ZFP that targets BCL11A (“BCL”); GFP (“GFP”); and a mock transfected control (“MCK”)) The bottom panels show the synergy score which describes the synergistic effects on the indicated ZFP repressor pairs.

FIG. 4 depicts a summary of exemplary results showing synergistic effects at various spacings as between repression domains; target site spacings as between ZFP-TF repressors; target site distance from the TSS; and strand bound by the ZFP-TF repressors. The top left panel shows synergistic effects at the indicated distance between the repression (KRAB) domains of the ZFP-TF repressors, where each Krab domain location is the position of the last targeted base of the C-terminal Zinc Finger. The bottom left panel shows synergistic effects at various indicated distances from the TSS, where the TSS distance is calculated as the distance from the TSS to the central position in the gap between the two ZFP-TF repressors. The top right panel shows synergistic effects where the ZFP-TF repressors bind to target sites separated by the indicated base pair gap. The bottom right panel shows synergistic effects when the individual ZFP-TF repressors bind to the indicated DNA stands (+/+, both ZFP-TFs target the sense strand; −/−, both ZFP-TFs target the antisense strand; +/− or −/+, one ZFP-TF targets the sense strand and one targets the antisense strand).

FIG. 5 depicts repression of tau expression using the indicated ZFP-TF repressors (ZFP designs shown in Table 1). For individual ZFP-TFs (top panel), each graph shows the 8 doses used (1000, 300, 100, 30, 10, 3, 1, 0.3 ng mRNA from left to right). For the genetic modulators comprising the two indicated ZFP-TFs, each graph shows the 8 doses used (300, 100, 30, 10, 3, 1, 0.3, 0.1 ng mRNA from left to right). All 6 single ZFP-TF repressors were also co-transfected and assessed for tau repression at 8 doses (100, 30, 10, 3, 1, 0.3, 0.1, 0.03 ng mRNA from left to right). The EC50 for each dose response curve is indicated in the upper right.

FIG. 6A through FIG. 6B depict off target effects and tau repression levels using the indicated ZFPs. FIG. 6A shows off-target events:52335 repressed 2 non-target genes and activated one off-target gene; 52389 activated one off-target site; and the pair of 52335 and 52389 activated one off-target site and repressed one off-target site. FIG. 6B are graphs depicting repression of tau following administration of the indicated ZFP-TFs. For individual ZFP-TFs (left and middle panel), each graph shows the 8 doses used (1000, 300, 100, 30, 10, 3, 1, 0.3 ng mRNA from left to right). For the genetic modulators comprising the two indicated ZFP-TFs (right panel), the graph shows the 8 doses used (300, 100, 30, 10, 3, 1, 0.3, 0.1 ng mRNA from left to right). qPCR analysis demonstrated that repressors comprising two ZFP-TF repressors repressed tau levels more than the individual repressors (0.012× wild-type levels).

FIG. 7A through FIG. 7C depicts tau repression in Neuro2A cells using multi-cistronic mRNA, the kinetics of repression following dosing with a synergistic ZFP-TF pair, and long-term silencing of the tau locus following transient delivery of ZFP genetic modulators. FIG. 7A shows repression using linked (multi-cistronic) and unlinked artificial TFs with codon-diversified variants of the Kox repression domain (designated nKox, mKox, and cKox for the N-terminal, Middle, or C-terminal position within the linked architecture, respectively). The results are shown using the indicated pairs administered in mRNA form as shown above the graph. The top panel is a schematic showing the potential configuration of three ZFPs linked by a tri-cistronic architecture with the indicated codon-diversified repression domains (nKOX, mKOX, cKOX) and linkages by the viral cleavage peptides T2A and P2A. The middle panels show results of tau repression with unlinked mRNA at doses of 300, 100, 30, 10, 3, 1, 0.3 and 0.1 ng mRNA (left to right in each graph) and the bottom panels show results of tau repression with bi-cistronic mRNA including the indicated linkers and repression domain at doses of 600, 200, 60, 20, 6, 2, 0.6, and 0.1 ng of mRNA (left to right in each graph). FIG. 7B shows tau expression levels at the indicated times following mRNA transfection. The top panel is a graph showing typical repression data for a repressor comprising ZFP-TFs 52322/52335 at 24 hours harvest post transfection. The bottom graphs show tau repression at 6 doses (300, 100, 30, 10, 3, and 1 ng of mRNA; left to right in each graph) over a time course at the indicated time points (24 hours, 48 hours, 64 hours, 72 hours and 136 hours). Also shown in the right-most bottom graph are negative transfection controls (a control ZFP that targets BCL11A (“B”); GFP (“G”); and a mock transfected controls (“M”)). FIG. 7C shows tau expression levels in Neuro2A cells at 1, 4, or 7 days post-transfection after transient ZFP delivery (3-dose mRNA transfection at 900/300/100 ng mRNA for the single factors and triple transfection at 300/100/30 ng mRNA each of 57890-KRAB, 52322-DNMT3A, and 57930-DNMT3L). The cells were cultured in growth-inhibited low serum media to arrest cell division.

FIG. 8A to FIG. 8C are graphs depicting tau expression and ZFP levels from in vivo of samples taken from control and treated nonhuman primate (NHP) subjects. FIG. 8A shows results from control subjects (NHP01, NHP02 and NHP03). FIG. 8B shows results from NHP subjects (NHP04, NHP05 and NHP06) treated with genetic repressors 65918 (“918”) and 57890 (“890”) carried by a single AAV2/9 vector where expression of the repressor (918 and 890) is driven by the synapsin (SYN1) promoter (“SYN1.918-890”). FIG. 8C shows results from subjects (NHP07 and NHP08) treated with genetic repressors 65918 (“918”) and 57890 (“890”) carried by a single AAV2/9 vector where expression of the repressor (918 and 890) is driven by a CMV promoter (“CMV.918-890”) (left panel). The top plot in each panel shows % normalized tau repression and the bottom plot in each panel shows ZFP levels (copies/ng mRNA).

FIG. 9A through FIG. 9C depict Tau repression in human iPS neurons. FIG. 9A shows that combinations of less active ZFPs exhibit synergy when used in combination (compare activity of the three proteins in cells treated with the single compounds as compared to the activity when two are used together. The human iPS derived neurons were treated with AAV6 comprising the ZFP-TF regulated by the Syn1 promoter wherein the cells were analyzed 19 days later. Cells were treated with 1E5 VG/cell, in 5-7 biological replicates. Note, **** indicates significance where p<0.0001. FIG. 9B depicts the changes in the transcriptome where with the ZFP-TFs used as single genetic modulators result in a slight repression of the MAPT gene, whereas when the two ZFP-TFs are used together, there is a much larger repression of MAPT expression. The plots depict the number of genes that are up or down regulated in the upper left corners, where the cut off for these reports is >2 fold change up or down. In these experiments, 19,959 coding transcripts were evaluated. “65918n” means that the 65918 ZFP-TF comprised the nKOX variant. “57890m” means that the 57890 ZFP-TF comprised the mKOX variant. FIG. 9C depicts the results found with the other ZFP-TF combination. “65920n” means that the 65920 ZFP-TF comprised the nKOX variant. “57890m” is the same as above.

FIG. 10 depicts results of repression of the mouse prion (Prnp) gene by exemplary ZFP-TFs (designated A to K) in Neuro2A cells transfected with mRNA encoding the ZFP repressors and the corresponding synergy score for each combination. The top two panels (“ZFP 1” and ZFP 2”) show results of the indicated ZFP-TF when used alone. The bottom panel shows the synergy score which describes the synergistic effects on the indicated ZFP repressor pairs (calculated as the ratio of expression levels obtained with the stronger ZFP when tested at 2× of its dose in the combination to the expression level obtained with the ZFP combination).

FIG. 11 depicts a summary showing synergistic effects of 130 combinations of ZFP-TFs targeting the mouse prion gene at various spacings between repression domains; target site spacings between ZFP-TF repressors; and target site distance from the TSS. The top panel shows synergistic effects where the ZFP-TF repressors bind to target sites separated by the indicated base pair gap. The middle panel shows synergistic effects at the indicated distance between the repression (KRAB) domains of the ZFP-TF repressors, where each KRAB domain location is the position of the last targeted base of the C-terminal zinc finger. The bottom panel shows synergistic effects at various indicated distances from the TSS, where the TSS distance is calculated as the distance from the TSS to the central position in the gap between the two ZFP-TF repressors.

FIG. 12 depicts results of repression of the human prion (PRNP) gene by exemplary ZFP-TFs (designated hA to hJ) in SK-N-MC cells transfected with mRNA encoding the ZFP repressors and the corresponding synergy score for each combination. The top two panels (“ZFP 1” and ZFP 2”) show results of the indicated ZFP-TF when used alone. The bottom panel shows the synergy score which describes the synergistic effects on the indicated ZFP repressor pairs (calculated as the ratio of expression levels obtained with the stronger ZFP when tested at 2× of its dose in the combination: the expression level obtained with the ZFP combination).

FIG. 13 depicts a summary showing synergistic effects of 130 combinations of ZFP-TFs targeting the human prion gene at various spacings between repression domains; target site spacings between ZFP-TF repressors; and target site distance from the TSS. The top panel shows synergistic effects where the ZFP-TF repressors bind to target sites separated by the indicated base pair gap. The middle panel shows synergistic effects at the indicated distance between the repression (KRAB) domains of the ZFP-TF repressors, where each KRAB domain location is the position of the last targeted base of the C-terminal zinc finger. The bottom panel shows synergistic effects at various indicated distances from the TSS, where the TSS distance is calculated as the distance from the TSS to the central position in the gap between the two ZFP-TF repressors.

FIG. 14 depicts results of repression of the human a-synuclein (SNCA) gene by exemplary ZFP-TFs (designated sA to sJ) in SK-N-MC cells transfected with mRNA encoding the ZFP repressors and the corresponding synergy score for each combination. The top two panels (“ZFP 1” and ZFP 2”) show results of the indicated ZFP-TF when used alone. The bottom panel shows the synergy score which describes the synergistic effects on the indicated ZFP repressor pairs (calculated as the ratio of expression levels obtained with the stronger ZFP when tested at 2× of its dose in the combination: the expression level obtained with the ZFP combination).

FIG. 15 depicts a summary showing synergistic effects of 132 combinations of ZFP-TFs targeting the human a-synuclein gene at various spacings between repression domains; target site spacings between ZFP-TF repressors; and target site distance from the TSS. The top panel shows synergistic effects where the ZFP-TF repressors bind to target sites separated by the indicated base pair gap. The middle panel shows synergistic effects at the indicated distance between the repression (KRAB) domains of the ZFP-TF repressors, where each KRAB domain location is the position of the last targeted base of the C-terminal zinc finger. The bottom panel shows synergistic effects at various indicated distances from the TSS, where the TSS distance is calculated as the distance from the TSS to the central position in the gap between the two ZFP-TF repressors.

### DETAILED DESCRIPTION

Disclosed herein are compositions and methods for modulating gene expression of a target gene with high specificity. The genetic modulators described herein include at least two artificial transcription factors, which provide synergistic (more than additive) effects as compared to individual artificial transcription factors. In particular, the compositions and methods described herein are used to modulate (e.g., repress or activate) the expression of any target gene. These genetic modulators may be used to modify gene expression in vivo such that the effects and/or symptoms of a disease associated with undesirable expression of the target gene is(are) reduced or eliminated.

For example, repressors as described herein can be used to reduce or eliminate the aggregation of tau or mutant Htt in the brain of a subject with a tauopathy (e.g., AD) or HD and reducing the symptoms of the disease.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al., MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526.

“TtAgo” is a prokaryotic Argonaute protein thought to be involved in gene silencing. TtAgo is derived from the bacteria Thermus thermophilus. See, e.g., Swarts et al., (2014) Nature 507(7491):258-261, G. Sheng et al., (2013) Proc. Natl. Acad. Sci. U.S.A. 111, 652). A “TtAgo system” is all the components required including, for example, guide DNAs for cleavage by a TtAgo enzyme. “Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

DNA-binding domains such as sgRNAs, zinc finger binding domains or TALE DNA binding domains can be “engineered” to bind to a predetermined nucleotide sequence, for example via design of a sgRNA that binds to a selected target site or by engineering (altering one or more amino acids) of the recognition helix region of a naturally occurring zinc finger protein or by engineering the RVDs of a TALE protein. Therefore, engineered zinc finger proteins or TALEs are proteins that are non-naturally occurring. Non-limiting examples of methods for engineering DNA-binding domains are design and selection. A “designed” zinc finger protein or TALE is a protein not occurring in nature whose design/composition results principally from rational criteria. Rational criteria for design include application of substitution rules and computerized algorithms for processing information in a database storing information of existing ZFP designs and binding data. A “selected” zinc finger protein or TALE is a protein not found in nature whose production results primarily from an empirical process such as phage display, interaction trap or hybrid selection. See, for example, U.S. Pat. Nos. 8,586,526; 6,140,081; 6,453,242; 6,746,838; 7,241,573; 6,866,997; 7,241,574; and 6,534,261; see also International Patent Publication No. WO 03/016496.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, where the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “multimerization domain”, (also referred to as a “dimerization domain” or “protein interaction domain”) is a domain incorporated at the amino, carboxy or amino and carboxy terminal regions of a ZFP TF or TALE TF. These domains allow for multimerization of multiple ZFP TF or TALE TF units such that larger tracts of trinucleotide repeat domains become preferentially bound by multimerized ZFP TFs or TALE TFs relative to shorter tracts with wild-type numbers of lengths. Examples of multimerization domains include leucine zippers. Multimerization domains may also be regulated by small molecules where the multimerization domain assumes a proper conformation to allow for interaction with another multimerization domain only in the presence of a small molecule or external ligand. In this way, exogenous ligands can be used to regulate the activity of these domains.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALE protein as described herein. Thus, gene inactivation may be partial or complete.

A “genetic modulator” refers to any molecule that alters the expression and/or sequence of one or more genes. Non-limiting examples of genetic modulators include transcription factors (such as artificial transcription factors as described herein) that bind to the target gene and alter its expression and nucleases that modify the sequence of the target gene, which in turn alters its expression (e.g., inactivation of the target via insertions and/or deletions). Thus, a genetic modulator may be a genetic repressor (that represses and/or inactivates gene expression) or a genetic activator.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion molecule in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to upregulate gene expression. ZFPs fused to domains capable of regulating gene expression are collectively referred to as “ZFP-TFs” or “zinc finger transcription factors”, while TALEs fused to domains capable of regulating gene expression are collectively referred to as “TALE-TFs” or “TALE transcription factors.” When a fusion polypeptide in which a ZFP DNA-binding domain is fused to a cleavage domain (a “ZFN” or “zinc finger nuclease”), the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site. When a fusion polypeptide in which a TALE DNA-binding domain is fused to a cleavage domain (a “TALEN” or “TALE nuclease”), the TALE DNA-binding domain and the cleavage domain are in operative linkage if, in the fusion polypeptide, the TALE DNA-binding domain portion is able to bind its target site and/or its binding site, while the cleavage domain is able to cleave DNA in the vicinity of the target site. With respect to a fusion molecule in which a Cas DNA-binding domain (e.g., single guide RNA) is fused to an activation domain, the Cas DNA-binding domain and the activation domain are in operative linkage if, in the fusion polypeptide, the Cas DNA-binding domain portion is able to bind its target site and/or its binding site, while the activation domain is able to up-regulate gene expression. When a fusion polypeptide in which a Cas DNA-binding domain is fused to a cleavage domain, the Cas DNA-binding domain and the cleavage domain are in operative linkage if, in the fusion polypeptide, the Cas DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al., (1989) Nature 340:245-246; U.S. Pat. No. 5,585,245 and International Patent Publication No. WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

The terms “synergy” and “additive” are used to refer to gene modulation effects achieved. When two or more artificial transcription factors modulate gene expression at levels higher than the individual artificial transcription factors and/or the expected (“additive”) modulation of the two or more artificial transcription factors used together, the modulation is said to exhibit synergy. “Synergy” includes functional synergy in which the individual components are all active at a given dose and cooperative synergy in which at least one of the individual artificial transcription factors of the genetic modular is inactive at a given dose. Synergy may be determined by any suitable means, for example by (1) calculating the ratio of the expected normalized expression of the target gene at the same dose for the strongest single artificial transcription factor to the observed normalized gene expression when the combination is used or (2) determining the ratio of expression levels obtained with the stronger ZFP-TF (at 2× of its dose used in the combination) to that obtained with the ZFP combination.

### Genetic Modulators

The genetic modulators described herein include two or more artificial transcription factors (e.g., repressors or activators), each artificial transcription factor (TF) comprising a DNA-binding domain and one or more functional domains. The genetic modulators described herein exhibit synergistic effects as compared to single transcription factors, including synergistic effects on specificity (limiting or eliminating modulation of off-target genes) and/or activity (amount of modulation). Thus, synergy is any increase in activity and/or specificity of more than about 1-fold, about 2-fold, about 3-fold, about 4-fold, about 5-fold, about 6-fold, about 7-fold, about 8-fold or more as compared to individual TFs (and/or expected additive effects).

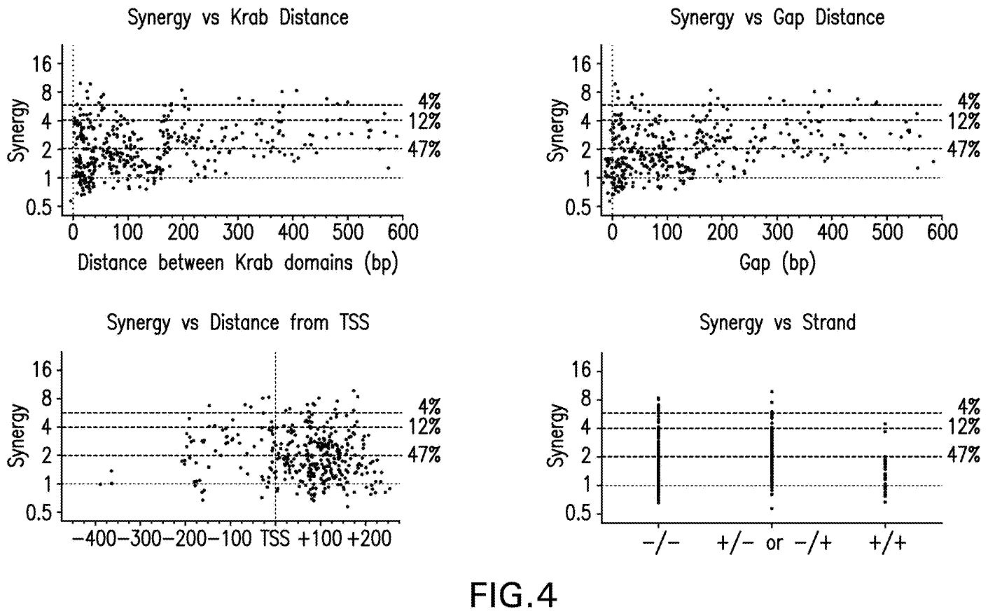

In any of the compositions described herein, the two or more artificial transcription factors can bind to target sites (via the DNA-binding domains of the TFs) that are between about 1 and about 600 (or any value therebetween) base pairs apart, preferably about 1 to about 300 (or any value therebetween) base pairs apart, and even more preferably about 1 to about 100 (or any value therebetween) base pairs apart. In certain embodiments, the components of the synergistic TF compositions bind to target sites that are approximately 1 to about 80 (or any value therebetween), approximately 160 to about 220 (or any value therebetween), approximately 260 to about 400 (or any value therebetween), or approximately 500 to about 600 (or any value therebetween) base pairs apart. See, e.g., FIGS. 4; 11; 13; and 15.

In any of the compositions described herein, the functional) domains (e.g., transcriptional activation or repression domains such as KRAB or DNMT) of the two or more artificial transcription factors (via the DNA-binding domains of the TFs) are positioned between about 1 and about 600 (or any value therebetween) base pairs apart from each other, preferably about 1 to about 300 (or any value therebetween) base pairs apart, and even more preferably about 1 to about 100 (or any value therebetween) base pairs apart. In certain embodiments, the functional domains of the synergistic TF compositions are positioned such that they are approximately 1 to about 80 (or any value therebetween), approximately 160 to about 220 (or any value therebetween), approximately 260 to about 400 (or any value therebetween), or approximately 500 to about 600 (or any value therebetween) base pairs apart from each other. See, e.g., FIG. 4; FIG. 11; FIG. 13; and FIG. 15.

The synergistic compositions described herein may bind to target sites anywhere in the target gene, including but not limited to coding sequences and adjacent or distal control elements (e.g., enhancers, promoters, etc.). In certain aspects, the TFs of the composition bind to target sites that are within 0-600 base pairs (or any value therebetween) on either side of the transcription start site (TSS). In certain embodiments, the TFs bind to target sites that are between the TSS and +200 (or any value therebetween) of the TSS. See, e.g., FIGS. 4; FIG. 11; FIG. 13; and FIG. 15.

Furthermore, the two or more TFs of the compositions described herein can bind to the same and/or different strands of the target site (e.g., endogenous gene). In certain embodiments, the synergistic composition comprises TFs that bind to the same antisense (−) or sense (+) strand. In other embodiments, the synergistic composition comprises TF that bind to different strands (+/− in either orientation). See, e.g., FIG. 4; FIG. 11; FIG. 13; and FIG. 15.

### DNA-Binding Domains

Any polynucleotide or polypeptide DNA-binding domain can be used in the compositions and methods disclosed herein, for example DNA-binding proteins (e.g., ZFPs or TALEs) or DNA-binding polynucleotides (e.g., single guide RNAs). The DNA-binding domains of the genetic modulator may be targeted to any gene of interest, including one or more genes aberrantly expressed in a disease or disorder. Two or more of the target sites recognized by the DNA-binding domain may be overlapping or non-overlapping. The target sites for two of the DNA-binding domains may be separated by up to about 600 or more base pairs and may be up to 300 or more base pairs from the transcription start site (on either side) of the target gene. In addition, when targeting double-stranded DNA, such as an endogenous genome, the DNA-binding domains of the artificial transcription factors may target the same or different stands (one or more to positive strand and/or one or more to negative strand). Further, the same or different DNA-binding domains may be used in the genetic modulators of the invention. Thus, genetic modulators (repressors) of any gene are described.

In certain embodiments, at least one DNA binding domain comprises a zinc finger protein. Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; 6,200,759; and International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536; and WO 03/016496.

ZFP DNA-binding domains include at least one zinc finger but can include a plurality of zinc fingers (e.g., 2, 3, 4, 5, 6 or more fingers). Usually, the ZFPs include at least three fingers. Certain of the ZFPs include four, five or six fingers, while some ZFPs include 8, 9, 10, 11 or 12 or more fingers. The ZFPs that include three fingers typically recognize a target site that includes 9 or 10 nucleotides; ZFPs that include four fingers typically recognize a target site that includes 12 to 14 nucleotides; while ZFPs having six fingers can recognize target sites that include 18 to 21 nucleotides. The ZFPs can also be fusion proteins that include one or more functional (regulatory) domains, which domains can be transcriptional activation or repression domains or other domains such as DNMT domains. The DNA binding domains fused to at least one regulatory (functional) domain and can be thought of as a ‘ZFP-TF’ architecture.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, and 8,772,453 incorporated by reference herein in their entireties.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

A ZFP can be operably associated (linked) to one or more transcriptional regulatory (e.g., repression domains) to form a ZFP-TF (e.g., repressor). Methods and compositions can also be used to increase the specificity of a ZFP for its intended target relative to other unintended cleavage sites, known as off-target sites for example by mutations to the ZFP backbone as described in U.S. Patent Publication No. 20180087072. Thus, genetic modulators described herein can comprise mutations in one or more of their DNA binding domain backbone regions and/or one or more mutations in their transcriptional regulatory domains. These ZFPs can include mutations to amino acid within the ZFP DNA binding domain (‘ZFP backbone’) that can interact non-specifically with phosphates on the DNA backbone, but they do not comprise changes in the DNA recognition helices. Thus, the invention includes mutations of cationic amino acid residues in the ZFP backbone that are not required for nucleotide target specificity. In some embodiments, these mutations in the ZFP backbone comprise mutating a cationic amino acid residue to a neutral or anionic amino acid residue. In some embodiments, these mutations in the ZFP backbone comprise mutating a polar amino acid residue to a neutral or non-polar amino acid residue. In preferred embodiments, mutations at made at position (−5), (−9) and/or position (−14) relative to the DNA binding helix. In some embodiments, a zinc finger may comprise one or more mutations at (−5), (−9) and/or (−14). In further embodiments, one or more zinc finger in a multi-finger zinc finger protein may comprise mutations in (−5), (−9) and/or (−14). In some embodiments, the amino acids at (−5), (−9) and/or (−14) (e.g. an arginine (R) or lysine (K)) are mutated to an alanine (A), leucine (L), Ser (S), Asp (N), Glu (E), Tyr (Y) and/or glutamine (Q).

Alternatively, the DNA-binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII are known. See also U.S. Pat. Nos. 5,420,032; 6,833,252; Belfort et al., (1997) Nucleic Acids Res. 25:3379-3388; Dujon et al., (1989) Gene 82:115-118; Perler et al., (1994) Nucleic Acids Res. 22, 1125-1127; Jasin (1996) Trends Genet. 12:224-228; Gimble et al., (1996) J. Mol. Biol. 263:163-180; Argast et al., (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al., (2002) Molec. Cell 10:895-905; Epinat et al., (2003) Nucleic Acids Res. 31:2952-2962; Ashworth et al., (2006) Nature 441:656-659; Paques et al., (2007) Current Gene Therapy 7:49-66; U.S. Patent Publication No. 2007/0117128.

In certain embodiments, the DNA-binding domain comprises a naturally occurring or engineered (non-naturally occurring) TAL effector (TALE) DNA binding domain. See, e.g., U.S. Pat. No. 8,586,526, incorporated by reference in its entirety herein. In certain embodiments, the TALE DNA-binding protein comprises binds to 12, 13, 14, 15, 16, 17, 18, 19, 20 or more contiguous nucleotides of a tau target site as shown in U.S. Publication No. 20180153921. The RVDs of the TALE DNA-binding protein that binds to a tau target site may be naturally occurring or non-naturally occurring RVDs. See, U.S. Pat. Nos. 8,586,526 and 9,458,205.

The plant pathogenic bacteria of the genus Xanthomonas are known to cause many diseases in important crop plants. Pathogenicity of Xanthomonas depends on a conserved type III secretion (T3 S) system which injects more than 25 different effector proteins into the plant cell. Among these injected proteins are transcription activator-like effectors (TALE) which mimic plant transcriptional activators and manipulate the plant transcriptome (see Kay et al., (2007) Science 318:648-651). These proteins contain a DNA binding domain and a transcriptional activation domain. One of the most well characterized TALEs is AvrBs3 from Xanthomonas campestgris pv. Vesicatoria (see Bonas et al., (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al., (2006) J Plant Physiol 163(3):256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al., (2007) Appl and Envir Micro 73(13):4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al., ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove (2009) Science 326:1501 and Boch et al., (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and NG binds to T. These DNA binding repeats have been assembled into proteins with new combinations and numbers of repeats, to make artificial transcription factors that are able to interact with new sequences. In addition, U.S. Pat. No. 8,586,526 and U.S. Patent Publication No. 2013/0196373, incorporated by reference in their entireties herein, describe TALEs with N-cap polypeptides, C-cap polypeptides (e.g., +63, +231 or +278) and/or novel (atypical) RVDs.

Exemplary TALEs are described in U.S. Pat. No. 8,586,526 and 9,458,205, incorporated by reference in their entireties.

In certain embodiments, the DNA binding domains include a dimerization and/or multimerization domain, for example a coiled-coil (CC) and dimerizing zinc finger (DZ). See, U.S. Patent Publication No. 2013/0253040.

In still further embodiments, the DNA-binding domain comprises a single-guide RNA of a CRISPR/Cas system, for example sgRNAs as disclosed in 20150056705.

Compelling evidence has recently emerged for the existence of an RNA-mediated genome defense pathway in archaea and many bacteria that has been hypothesized to parallel the eukaryotic RNAi pathway (for reviews, see Godde and Bickerton, 2006. J. Mol. Evol. 62:718-729; Lillestol et al., 2006. Archaea 2:59-72; Makarova et al., 2006. Biol. Direct 1:7.; Sorek et al., 2008. Nat. Rev. Microbiol. 6:181-186). Known as the CRISPR-Cas system or prokaryotic RNAi (pRNAi), the pathway is proposed to arise from two evolutionarily and often physically linked gene loci: the CRISPR (clustered regularly interspaced short palindromic repeats) locus, which encodes RNA components of the system, and the cas (CRISPR-associated) locus, which encodes proteins (Jansen et al., 2002. Mol. Microbiol. 43:1565-1575; Makarova et al., 2002. Nucleic Acids Res. 30:482-496; Makarova et al., 2006. Biol. Direct 1:7; Haft et al., 2005. PLoS Comput. Biol. 1:e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (Makarova et al., 2006. Biol. Direct 1:7). The CRISPR-associated (cas) genes are often associated with CRISPR repeat-spacer arrays. More than forty different Cas protein families have been described. Of these protein families, Casl appears to be ubiquitous among different CRISPR/Cas systems. Particular combinations of cas genes and repeat structures have been used to define 8 CRISPR subtypes (E. coli, Y. pest, N. meni, D. vulg, T neap, H; mari, A; pern, and M. tube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

The Type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system.

Type II CRISPR systems have been found in many different bacteria. BLAST searches on publicly available genomes by Fonfara et al., ((2013) Nuc Acid Res 42(4):2377-2590) found Cas9 orthologs in 347 species of bacteria. Additionally, this group demonstrated in vitro CRISPR/Cas cleavage of a DNA target using Cas9 orthologs from S. pyogenes, S. mutans, S. therophilus, C. jejuni, N. meningitides, P. multocida and F. novicida. Thus, the term “Cas9” refers to an RNA guided DNA nuclease comprising a DNA binding domain and two nuclease domains, where the gene encoding the Cas9 may be derived from any suitable bacteria.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The Cas 9 nuclease can be engineered such that only one of the nuclease domains is functional, creating a Cas nickase (see Jinek et al., (2012) Science 337:816). Nickases can be generated by specific mutation of amino acids in the catalytic domain of the enzyme, or by truncation of part or all of the domain such that it is no longer functional. Since Cas 9 comprises two nuclease domains, this approach may be taken on either domain. A double strand break can be achieved in the target DNA by the use of two such Cas 9 nickases. The nickases will each cleave one strand of the DNA and the use of two will create a double strand break.

The requirement of the crRNA-tracrRNA complex can be avoided by use of an engineered “single-guide RNA” (sgRNA) that comprises the hairpin normally formed by the annealing of the crRNA and the tracrRNA (see Jinek et al., ibid and Cong et al., (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam et al., Stem Cells and Development 22(4):595-610 (2013)) and has been useful for zebrafish embryo genomic editing in vivo (see Hwang et al., (2013) Nature Biotechnology 31 (3):227) with editing efficiencies similar to ZFNs and TALENs.

The primary products of the CRISPR loci appear to be short RNAs that contain the invader targeting sequences, and are termed guide RNAs or prokaryotic silencing RNAs (psiRNAs) based on their hypothesized role in the pathway (Makarova et al., 2006. Biol. Direct 1: 7; Hale et al., 2008. RNA, 14: 2572-2579). RNA analysis indicates that CRISPR locus transcripts are cleaved within the repeat sequences to release ^{˜}60- to 70-nt RNA intermediates that contain individual invader targeting sequences and flanking repeat fragments (Tang et al., 2002. Proc. Natl. Acad. Sci. 99: 7536-7541; Tang et al., 2005. Mol. Microbiol. 55: 469-481; Lillestol et al., 2006. Archaea 2: 59-72; Brouns et al., 2008. Science 321: 960-964; Hale et al., 2008. RNA, 14: 2572-2579). In the archaeon Pyrococcus furiosus, these intermediate RNAs are further processed to abundant, stable ^{˜}35- to 45-nt mature psiRNAs (Hale et al., 2008. RNA, 14: 2572-2579).

The requirement of the crRNA-tracrRNA complex can be avoided by use of an engineered “single-guide RNA” (sgRNA) that comprises the hairpin normally formed by the annealing of the crRNA and the tracrRNA (see Jinek et al., (2012) Science 337:816 and Cong et al., (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam, ibid) and has been useful for zebrafish embryo genomic editing in vivo (see Hwang et al., (2013) Nature Biotechnology 31 (3):227) with editing efficiencies similar to ZFNs and TALENs.

Chimeric or sgRNAs can be engineered to comprise a sequence complementary to any desired target. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In certain embodiments, the sgRNA comprises a sequence that binds to 12, 13, 14, 15, 16, 17, 18, 19, 20 or more contiguous nucleotides of a tau target site as shown in U.S. Publication No. 20180153921. In some embodiments, the RNAs comprise 22 bases of complementarity to a target and of the form G[n19], followed by a protospacer-adjacent motif (PAM) of the form NGG or NAG for use with a S. pyogenes CRISPR/Cas system. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence the conforms to the G[n20]GG formula. Along with the complementarity region, an sgRNA may comprise additional nucleotides to extend to tail region of the tracrRNA portion of the sgRNA (see Hsu et al., (2013) Nature Biotech doi:10.1038/nbt.2647). Tails may be of +67 to +85 nucleotides, or any number therebetween with a preferred length of +85 nucleotides. Truncated sgRNAs may also be used, “tru-gRNAs” (see Fu et al., (2014) Nature Biotech 32(3):279). In tru-gRNAs, the complementarity region is diminished to 17 or 18 nucleotides in length.

Further, alternative PAM sequences may also be utilized, where a PAM sequence can be NAG as an alternative to NGG (Hsu 2013, ibid) using a S. pyogenes Cas9. Additional PAM sequences may also include those lacking the initial G (Sander and Joung (2014) Nature Biotech 32(4):347). In addition to the S. pyogenes encoded Cas9 PAM sequences, other PAM sequences can be used that are specific for Cas9 proteins from other bacterial sources. For example, the PAM sequences shown below (adapted from Sander and Joung, ibid, and Esvelt et al., (2013) Nat Meth 10(11):1116) are specific for these Cas9 proteins:

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |

Thus, a suitable target sequence for use with a S. pyogenes CRISPR/Cas system can be chosen according to the following guideline: [n17, n18, n19, or n20](G/A)G. Alternatively the PAM sequence can follow the guideline G[n17, n18, n19, n20](G/A)G. For Cas9 proteins derived from non-S. pyogenes bacteria, the same guidelines may be used where the alternate PAMs are substituted in for the S. pyogenes PAM sequences.

Most preferred is to choose a target sequence with the highest likelihood of specificity that avoids potential off target sequences. These undesired off target sequences can be identified by considering the following attributes: i) similarity in the target sequence that is followed by a PAM sequence known to function with the Cas9 protein being utilized; ii) a similar target sequence with fewer than three mismatches from the desired target sequence; iii) a similar target sequence as in ii), where the mismatches are all located in the PAM distal region rather than the PAM proximal region (there is some evidence that nucleotides 1-5 immediately adjacent or proximal to the PAM, sometimes referred to as the ‘seed’ region (Wu et al., (2014) Nature Biotech doi:10.1038/nbt2889) are the most critical for recognition, so putative off target sites with mismatches located in the seed region may be the least likely be recognized by the sg RNA); and iv) a similar target sequence where the mismatches are not consecutively spaced or are spaced greater than four nucleotides apart (Hsu 2014, ibid). Thus, by performing an analysis of the number of potential off target sites in a genome for whichever CRIPSR/Cas system is being employed, using these criteria above, a suitable target sequence for the sgRNA may be identified.

In some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al. (2015) Genom Bio 16:251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1are 5′ -TTN-3′ and 5′ -CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term ‘“Cas” includes both Cas9 and Cfp1 proteins. Thus, as used herein, a “CRISPR/Cas system” refers both CRISPR/Cas and/or CRISPR/Cfp1 systems, including both nuclease, nickase and/or transcription factor systems.

In some embodiments, other Cas proteins may be used. Some exemplary Cas proteins include Cas9, Cpf1 (also known as Cas12a), C2c1, C2c2 (also known as Cas13a), C2c3, Cas1, Cas2, Cas4, CasX and CasY; and include engineered and natural variants thereof (Burstein et al. (2017) Nature 542:237-241) for example HF1/spCas9 (Kleinstiver et al. (2016) Nature 529: 490-495; Cebrian-Serrano and Davies (2017) Mamm Genome (2017) 28(7):247-261); split Cas9 systems (Zetsche et al. (2015) Nat Biotechnol 33(2):139-142), trans-spliced Cas9 based on an intein-extein system (Troung et al. (2015) Nucl Acid Res 43(13):6450-8); mini-SaCas9 (Ma et al. (2018) ACS Synth Biol 7(4):978-985). Thus, in the methods and compositions described herein, it is understood that the term ‘“Cas” includes all Cas variant proteins, both natural and engineered. Thus, as used herein, a “CRISPR/Cas system” refers to any CRISPR/Cas system, including both nuclease, nickase and/or transcription factor systems.

In certain embodiments, the Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to specific genes (including safe harbor genes) are disclosed for example, in U.S. Publication No. 2015/0056705.

Thus, the nuclease comprises a DNA-binding domain in that specifically binds to a target site in any gene into which it is desired to insert a donor (transgene) in combination with a nuclease domain that cleaves DNA.

### Functional Domains

The DNA-binding domains may be fused to or otherwise associated with one or more functional domains to form artificial transcription factors as described herein. In certain embodiments, the methods employ fusion molecules comprising at least one DNA-binding molecule (e.g., ZFP, TALE or single guide RNA) and a heterologous regulatory (functional) domain (or functional fragment thereof).

In certain embodiments, the functional domain of the artificial transcription factor of the genetic modulator comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP16 activation domain (see, e.g., Hagmann et al., J. Virol. 71, 5952-5962 (1997)) nuclear hormone receptors (see, e.g., Torchia et al., Curr. Opin. Cell. Biol. 10:373-383 (1998)); the p65 subunit of nuclear factor kappa B (Bitko & Barik, J Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Sp1, AP-2, and CTF1 (Seipel et al., EMBO J. 4961-4968 (1992) as well as p300, CBP, PCAF, SRC1 PvALF, AtHD2A and ERF-2. See, for example, Robyr et al., (2000) Mol. Endocrinol. 14:329-347; Collingwood et al., (1999) J. Mol. Endocrinol. 23:255-275; Leo et al., (2000) Gene 245:1-11; Manteuffel-Cymborowska (1999) Acta Biochim. Pol. 46:77-89; McKenna et al., (1999) J. Steroid Biochem. Mol. Biol. 69:3-12; Malik et al., (2000) Trends Biochem. Sci. 25:277-283; and Lemon et al., (1999) Curr. Opin. Genet. Dev. 9:499-504. Additional exemplary activation domains include, but are not limited to, OsGAI, HALF-1, C1, AP1, ARF-5,-6,-7, and -8, CPRF1, CPRF4, MYC-RP/GP, and TRAB1. See, for example, Ogawa et al., (2000) Gene 245:21-29; Okanami et al., (1996) Genes Cells 1:87-99; Goff et al., (1991) Genes Dev. 5:298-309; Cho et al., (1999) Plant Mol. Biol. 40:419-429; Ulmason et al., (1999) Proc. Natl. Acad. Sci. USA 96:5844-5849; Sprenger-Haussels et al., (2000) Plant J. 22:1-8; Gong et al., (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al., (1999) Cell 99:451-454; Tyler et al., (1999) Cell 99:443-446; Knoepfler et al., (1999) Cell 99:447-450; and Robertson et al., (2000) Nature Genet. 25:338-342. Additional exemplary repression domains include, but are not limited to, ROM2 and AtHD2A. See, for example, Chem et al., (1996) Plant Cell 8:305-321; and Wu et al., (2000) Plant J. 22:19-27.

In some instances, the domain is involved in epigenetic regulation of a chromosome. In some embodiments, the domain is a histone acetyltransferase (HAT), e.g. type-A, nuclear localized such as MYST family members MOZ, Ybf2/Sas3, MOF, and Tip60, GNAT family members Gcn5 or pCAF, the p300 family members CBP, p300 or Rtt109 (Berndsen and Denu (2008) Curr Opin Struct Biol 18(6):682-689). In other instances the domain is a histone deacetylase (HDAC) such as the class I (HDAC-1, 2, 3, and 8), class II (HDAC IIA (HDAC-4, 5, 7 and 9), HDAC IIB (HDAC 6 and 10)), class IV (HDAC-11), class III (also known as sirtuins (SIRTs); SIRT1-7) (see Mottamal et al., (2015) Molecules 20(3):3898-3941). Another domain that is used in some embodiments is a histone phosphorylase or kinase, where examples include MSK1, MSK2, ATR, ATM, DNA-PK, Bub 1, VprBP, IKK-α, PKCβ1, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dot1, PRMT 1/6, PRMT 5/7, PR-Set7 and Suv4-20h. Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, Ill.) Catalogue. Methods and compositions for making fusions between a minor groove binder and a polypeptide have been described. Mapp et al., (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

The fusion molecule may be formulated with a pharmaceutically acceptable carrier, as is known to those of skill in the art. See, for example, Remington's Pharmaceutical Sciences, 17th ed., 1985; and co-owned International Patent Publication No. WO 00/42219.

The functional component/domain of a fusion molecule can be selected from any of a variety of different components capable of influencing transcription of a gene once the fusion molecule binds to a target sequence via its DNA binding domain. Hence, the functional component can include, but is not limited to, various transcription factor domains, such as activators, repressors, co-activators, co-repressors, and silencers.

In certain embodiments, the fusion molecule comprises a DNA-binding domain and a nuclease domain to create functional entities that are able to recognize their intended nucleic acid target through their engineered (ZFP or TALE or sgRNA) DNA binding domains and create nucleases (e.g., zinc finger nuclease or TALE nucleases or CRISPR/Cas nucleases) cause the DNA to be cut near the DNA binding site via the nuclease activity. This cleavage results in inactivation (repression) of a tau gene. Thus, genetic repressors also include nucleases.

Thus, the methods and compositions described herein are broadly applicable and may involve any nuclease of interest. Non-limiting examples of nucleases include meganucleases, TALENs and zinc finger nucleases. The nuclease may comprise heterologous DNA-binding and cleavage domains (e.g., zinc finger nucleases; TALENs; meganuclease DNA-binding domains with heterologous cleavage domains, sgRNAs in association with nuclease domains) or, alternatively, the DNA-binding domain of a naturally-occurring nuclease may be altered to bind to a selected target site (e.g., a meganuclease that has been engineered to bind to site different than the cognate binding site).

The nuclease domain may be derived from any nuclease, for example any endonuclease or exonuclease. Non-limiting examples of suitable nuclease (cleavage) domains that may be fused to DNA-binding domains as described herein include domains from any restriction enzyme, for example a Type IIS Restriction Enzyme (e.g., FokI). In certain embodiments, the cleavage domains are cleavage half-domains that require dimerization for cleavage activity. See, e.g., U.S. Pat. Nos. 8,586,526; 8,409,861; and 7,888,121, incorporated by reference in their entireties herein. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing.

The nuclease domain may also be derived any meganuclease (homing endonuclease) domain with cleavage activity may also be used with the nucleases described herein, including but not limited to I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

In certain embodiments, the nuclease comprises a compact TALEN (cTALEN). These are single chain fusion proteins linking a TALE DNA binding domain to a TevI nuclease domain. The fusion protein can act as either a nickase localized by the TALE region, or can create a double strand break, depending upon where the TALE DNA binding domain is located with respect to the meganuclease (e.g., TevI) nuclease domain (see Beurdeley et al., (2013) Nat Comm: 1-8 DOI:10.1038/ncomms2782).

In other embodiments, the TALE-nuclease is a mega TAL. These mega TAL nucleases are fusion proteins comprising a TALE DNA binding domain and a meganuclease cleavage domain. The meganuclease cleavage domain is active as a monomer and does not require dimerization for activity. (See Boissel et al., (2013) Nucl Acid Res:1-13, doi:10.1093/nar/gkt1224).

In addition, the nuclease domain of the meganuclease may also exhibit DNA-binding functionality. Any TALENs may be used in combination with additional TALENs (e.g., one or more TALENs (cTALENs or FokI-TALENs) with one or more mega-TALs) and/or ZFNs.

In addition, cleavage domains may include one or more alterations as compared to wild-type, for example for the formation of obligate heterodimers that reduce or eliminate off-target cleavage effects. See, e.g., U.S. Pat. Nos. 7,914,796; 8,034,598; and 8,623,618, incorporated by reference in their entireties herein.

Nucleases as described herein may generate double- or single-stranded breaks in a double-stranded target (e.g., gene). The generation of single-stranded breaks (“nicks”) is described, for example in U.S. Pat. Nos. 8,703,489 and 9,200,266, incorporated herein by reference which describes how mutation of the catalytic domain of one of the nucleases domains results in a nickase.

Thus, a nuclease (cleavage) domain or cleavage half-domain can be any portion of a protein that retains cleavage activity, or that retains the ability to multimerize (e.g., dimerize) to form a functional cleavage domain.

Alternatively, nucleases may be assembled in vivo at the nucleic acid target site using so-called “split-enzyme” technology (see e.g. U.S. Patent Publication No. 2009/0068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in U.S. Publication No. 2009/0111119. Nuclease expression constructs can be readily designed using methods known in the art.

Expression of the fusion proteins (or component thereof) may be under the control of a constitutive promoter or an inducible promoter, for example the galactokinase promoter which is activated (de-repressed) in the presence of raffinose and/or galactose and repressed in presence of glucose. Non-limiting examples of preferred promoters include the neural specific promoters NSE, CMV, Synapsin, CAMKiia and MECPs. Non-limiting examples of ubiquitous promoters include CAS and Ubc. Further embodiments include the use of self-regulating promoters (via the inclusion of high affinity binding sites for the DNA-binding domain) as described in U.S. Patent Publication No. 2015/0267205.

### Delivery

The proteins and/or polynucleotides (e.g., genetic modulators) and compositions comprising the proteins and/or polynucleotides described herein may be delivered to a target cell by any suitable means including, for example, by injection of proteins, via mRNA and/or using an expression construct (e.g., plasmid, lentiviral vector, AAV vector, Ad vector, etc.). In preferred embodiments, the repressor is delivered using an AAV vector, including but not limited to AAV2/6 or AAV2/9 (see U.S. Pat. No. 7,198,951), an AAV vector as described in U.S. Pat. No. 9,585,971.

Methods of delivering proteins comprising zinc finger proteins as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 8,586,526; 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more DNA-binding protein-encoding sequences. Thus, when one or more modulators (e.g., repressors) are introduced into the cell, the sequences encoding the protein components and/or polynucleotide components may be carried on the same vector or on different vectors. When multiple vectors are used, each vector may comprise a sequence encoding one or multiple modulators (e.g., repressors) or components thereof. In preferred embodiments, the vector system is an AAV vector, for example AAV6 or AAV9 or an AAV variant described in U.S. Pat. No. 9,585,971 or U.S. Publication No. 20170119906.

Conventional viral and non-viral based gene transfer methods can be used to introduce nucleic acids encoding engineered modulators in cells (e.g., mammalian cells) and target tissues. Such methods can also be used to administer nucleic acids encoding such repressors (or components thereof) to cells in vitro. In certain embodiments, nucleic acids encoding the repressors are administered for in vivo or ex vivo gene therapy uses. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, naked RNA, artificial virions, and agent-enhanced uptake of DNA. Sonoporation using, e.g., the Sonitron 2000 system (Rich-Mar) can also be used for delivery of nucleic acids. In a preferred embodiment, one or more nucleic acids are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., TransfectamTM and Lipofectin™ and Lipofectamine™ RNAiMAX). Cationic and neutral lipids that are suitable for efficient receptor-recognition lipofection of polynucleotides include those of Felgner, International Patent Publication Nos. WO 91/17424 and WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr etal., Bioconjugate Chem. 5:382-389 (1994); Remy etal., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGenelC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al., (2009) Nature Biotechnology 27(7):643).

The use of RNA or DNA viral based systems for the delivery of nucleic acids encoding engineered ZFPs, TALEs or CRISPR/Cas systems take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs, TALEs or CRISPR/Cas systems include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon mouse leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); International Patent Publication NO. WO 1994/026877).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; International Patent Publication No. WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV approximately 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6, AAV8, AAV 8.2, AAV9, and AAV rh10 and pseudotyped AAV such as AAV2/8, AAV2/5, AAV2/9 and AAV2/6 can also be used in accordance with the present invention. Novel AAV serotypes capable of crossing the blood-brain barrier can also be used in accordance with the present invention (see e.g. U.S. Pat. No. 9,585,971). In preferred embodiments, an AAV9 vector (including variants and pseudotypes of AAV9) is used.

Replication-deficient recombinant adenoviral vectors (Ad) can be produced at high titer and readily infect a number of different cell types. Most adenovirus vectors are engineered such that a transgene replaces the Ad E1a, E1b, and/or

E3 genes; subsequently the replication defective vector is propagated in human 293 cells that supply deleted gene function in trans. Ad vectors can transduce multiple types of tissues in vivo, including nondividing, differentiated cells such as those found in liver, kidney and muscle. Conventional Ad vectors have a large carrying capacity. An example of the use of an Ad vector in a clinical trial involved polynucleotide therapy for antitumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and Ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney mouse leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, intrathecal, intracisternal, intracerebroventricular, or intracranial infusion, including direct injection into the brain including into any region of the brain such as the hippocampus, cortex, striatum, etc.) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

In certain embodiments, the compositions as described herein (e.g., polynucleotides and/or proteins) are delivered directly in vivo. The compositions (cells, polynucleotides and/or proteins) may be administered directly into the central nervous system (CNS), including but not limited to direct injection into the brain or spinal cord. One or more areas of the brain may be targeted, including but not limited to, the hippocampus, the substantia nigra, the nucleus basalis of Meynert (NBM), the striatum and/or the cortex. Alternatively or in addition to CNS delivery, the compositions may be administered systemically (e.g., intravenous, intraperitoneal, intracardial, intramuscular, subdermal, intrathecal, intracisternal, intracerebroventricular and/or intracranial infusion). Methods and compositions for delivery of compositions as described herein directly to a subject (including directly into the CNS) include but are not limited to direct injection (e.g., stereotactic injection) via needle assemblies. Such methods are described, for example, in U.S. Pat. Nos. 7,837,668 and 8,092,429, relating to delivery of compositions (including expression vectors) to the brain and U.S. Patent Publication No. 2006/0239966, incorporated herein by reference in their entireties.

The effective amount to be administered will vary from patient to patient and according to the mode of administration and site of administration. Accordingly, effective amounts are best determined by the physician administering the compositions and appropriate dosages can be determined readily by one of ordinary skill in the art. After allowing sufficient time for integration and expression (typically 4-15 days, for example), analysis of the serum or other tissue levels of the therapeutic polypeptide and comparison to the initial level prior to administration will determine whether the amount being administered is too low, within the right range or too high. Suitable regimes for initial and subsequent administrations are also variable, but are typified by an initial administration followed by subsequent administrations if necessary. Subsequent administrations may be administered at variable intervals, ranging from daily to annually to every several years. In certain embodiments,

[symbol]

To deliver ZFPs using adeno-associated viral (AAV) vectors directly to the human brain, a dose range of 1×10¹-5×10¹⁵ (or any value therebetween) vector genome per striatum can be applied. As noted, dosages may be varied for other brain structures and for different delivery protocols. Methods of delivering AAV vectors directly to the brain are known in the art. See, e.g., U.S. Pat. Nos. 9,089,667; 9,050,299; 8,337,458; 8,309,355; 7,182,944; 6,953,575; and 6,309,634.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with at least one modulator (e.g., repressor) or component thereof and re-infused back into the subject organism (e.g., patient). In a preferred embodiment, one or more nucleic acids of the modulator (e.g., repressor) are delivered using AAV9. In other embodiments, one or more nucleic acids of the modulator (e.g., repressor) are delivered as mRNA. Also preferred is the use of capped mRNAs to increase translational efficiency and/or mRNA stability. Especially preferred are ARCA (anti-reverse cap analog) caps or variants thereof. See U.S. Pat. Nos. 7,074,596 and 8,153,773, incorporated by reference herein in their entireties. Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-γ and TNF-α are known (see Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

Stem cells that have been modified may also be used in some embodiments. For example, neuronal stem cells that have been made resistant to apoptosis may be used as therapeutic compositions where the stem cells also contain the ZFP TFs of the invention. Resistance to apoptosis may come about, for example, by knocking out BAX and/or BAK using BAX- or BAK-specific TALENs or ZFNs (see, U.S. Pat. No. 8,597,912) in the stem cells, or those that are disrupted in a caspase, again using caspase-6 specific ZFNs for example. These cells can be transfected with the ZFP TFs or TALE TFs that are known to regulate a target gene.

Vectors (e.g., retroviruses, adenoviruses, liposomes, etc.) containing therapeutic ZFP nucleic acids can also be administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Methods for introduction of DNA into hematopoietic stem cells are disclosed, for example, in U.S. Pat. No. 5,928,638. Vectors useful for introduction of transgenes into hematopoietic stem cells, e.g., CD34⁺ cells, include adenovirus Type 35.

Vectors suitable for introduction of transgenes into immune cells (e.g., T-cells) include non-integrating lentivirus vectors. See, for example, Ory et al., (1996) Proc. Natl. Acad. Sci. USA 93:11382-11388; Dull et al., (1998) J. Virol. 72:8463-8471; Zuffery et al., (1998) J. Virol. 72:9873-9880; Follenzi et al., (2000) Nature Genetics 25:217-222.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

As noted above, the disclosed methods and compositions can be used in any type of cell including, but not limited to, prokaryotic cells, fungal cells, Archaeal cells, plant cells, insect cells, animal cells, vertebrate cells, mammalian cells and human cells. Suitable cell lines for protein expression are known to those of skill in the art and include, but are not limited to COS, CHO (e.g., CHO-S, CHO-K1, CHO-DG44, CHO-DUXB11), VERO, MDCK, WI38, V79, B14AF28-G3, BHK, HaK, NSO, SP2/0-Ag14, HeLa, HEK293 (e.g., HEK293-F, HEK293-H, HEK293-T), perC6, insect cells such as Spodoptera fugiperda (Sf), and fungal cells such as Saccharomyces, Pischia and Schizosaccharomyces. Progeny, variants and derivatives of these cell lines can also be used. In a preferred embodiment, the methods and composition are delivered directly to a brain cell, for example in the striatum.

### Models of CNS Disorders

Studies of CNS disorders can be carried out in animal model systems such as non-human primates (e.g., Parkinson's Disease (Johnston and Fox (2015) Curr Top Behav Neurosci 22: 221-35); Amyotrophic lateral sclerosis (Jackson et al., (2015) J. Med Primatol: 44(2):66-75), Huntington's Disease (Yang et al., (2008) Nature 453(7197):921-4); Alzheimer's Disease (Park et al., (2015) Int J Mot Sci 16(2):2386-402); Seizure (Hsiao et al., (2016) E Bio Med 9:257-77), canines (e.g. MPS VII (Gurda et al., (2016) Mol Ther 24(2):206-216); Alzheimer's Disease (Schutt et al., (2016) J Alzheimers Dis 52(2):433-49); Seizure (Varatharajah et al., (2017) Int J Neural Syst 27(1):1650046) and mice (e.g. Seizure (Kadiyala et al., (2015) Epilepsy Res 109:183-96); Alzheimer's Disease (Li et al., (2015) J Alzheimers Dis Parkin 5(3) doi 10:4172/2161-0460), (review: Webster et al., (2014) Front Genet 5 art 88, doi:10.3389f/gene.2014.00088). These models may be used even when there is no animal model that completely recapitulates a CNS disease as they may be useful for investigating specific symptom sets of a disease. The models may be helpful in determining efficacy and safety profiles of a therapeutic methods and compositions (genetic repressors) described herein.

### Applications

Genetic modulators (e.g., repressors) as described herein comprising a plurality of artificial transcription factors can be used for any application in which specific modulation of gene expression is desired. These applications include therapeutic methods in which at least one genetic modulator is administered to a subject using a viral (e.g., AAV) or non-viral vector and used to modulate the expression of a target gene within the subject. The modulation can be in the form of repression, for example, repression of gene expression that is contributing to a disease state (e.g., Htt in HD, mutant C9ORF72 in ALS, SNCA in PD and DLB, tau in AD, PRNP in prion disease). Alternatively, the modulation can be in the form of activation when activation of expression or increased expression of an endogenous cellular gene can ameliorate a diseased state. As noted above, for such applications, the nucleic acids encoding the genetic modulators described herein are formulated with a pharmaceutically acceptable carrier as a pharmaceutical composition.

The genetic modulators, or vectors encoding them, alone or in combination with other suitable components (e.g. liposomes, nanoparticles or other components known in the art), can be made into aerosol formulations (i.e., they can be “nebulized”) to be administered via inhalation. Aerosol formulations can be placed into pressurized acceptable propellants, such as dichlorodifluoromethane, propane, nitrogen, and the like. Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Compositions can be administered, for example, by intravenous infusion, orally, topically, intraperitoneally, intravesically, retro-orbitally (RO), intracranially (e.g., to any area of the brain including but not limited to the hippocampus and/or cortex) or intrathecally. The formulations of compounds can be presented in unit-dose or multi-dose sealed containers, such as ampules and vials. Injection solutions and suspensions can be prepared from sterile powders, granules, and tablets of the kind previously described.

The dose administered to a patient should be sufficient to provide a beneficial therapeutic response in the patient over time. The dose is determined by the efficacy and K_{d} of the particular genetic modulator employed, the target cell, and the condition of the patient, as well as the body weight or surface area of the patient to be treated. The size of the dose also is determined by the existence, nature, and extent of any adverse side-effects that accompany the administration of a particular compound or vector in a particular patient.

The following Examples relate to exemplary embodiments of the present disclosure in which the genetic modulator comprises at least two zinc finger proteins that bind to a target gene. It will be appreciated that this is for purposes of exemplification only and that genetic modulators (e.g., repressors) for any target gene can be used, including, but not limited to, TALE-TFs, a CRISPR/Cas system, additional ZFPs, ZFNs, TALENs, additional CRISPR/Cas systems, homing endonucleases (meganucleases) with engineered DNA-binding domains. It will be apparent that these modulators can be readily obtained using methods known to the skilled artisan to bind to the target sites as exemplified below. Similarly, the following Examples relate to exemplary embodiments in which the delivery vehicle is any AAV vector but it will apparent that any viral (Ad, LV, etc.) or non-viral (plasmid, mRNA, etc.) can be used to deliver the modulators described herein.

Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. All publications and other references mentioned herein are incorporated by reference in their entirety. Although a number of documents are cited herein, this citation does not constitute an admission that any of these documents forms part of the common general knowledge in the art. As used herein, the term “approximately” or “about” as applied to one or more values of interest refers to a value that is similar to a stated reference value. In certain embodiments, the term refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context.

### EXAMPLES

### Example 1: Synergistic ZFP-TF Repressors

Compositions comprising synergistic ZFP-TF repressors were identified by screening panels of ZFP-TFs individually and in various combinations.

### A. Tau (MAPT)

A screen of approximately 185 zinc finger proteins was conducted as described in U.S. Publication No. 20180153921. ZFP-TFs comprising the ZFPs and a repression domain were also tested and found to repress expression. In addition, repression by the individual ZFPs was compared to various pairs combined and tested.

ZFP repressors were evaluated individually or in pairs for repression of tau in mouse Neuro2A (N2A) cells as follows. In brief, 3 different doses (about 30, 10 or 3 ng) of many different individual ZFP-TF and pairwise combinations of ZFP-TFs encoding mRNA were transfected into about 100,000 Neuro2A cells. ZFP TFs were transfected into mouse Neuro2a cells. After about 24 hours, total RNA was extracted and the expression of MAPT and two reference genes (ATP5b, RPL38) was monitored using real-time RT-qPCR.

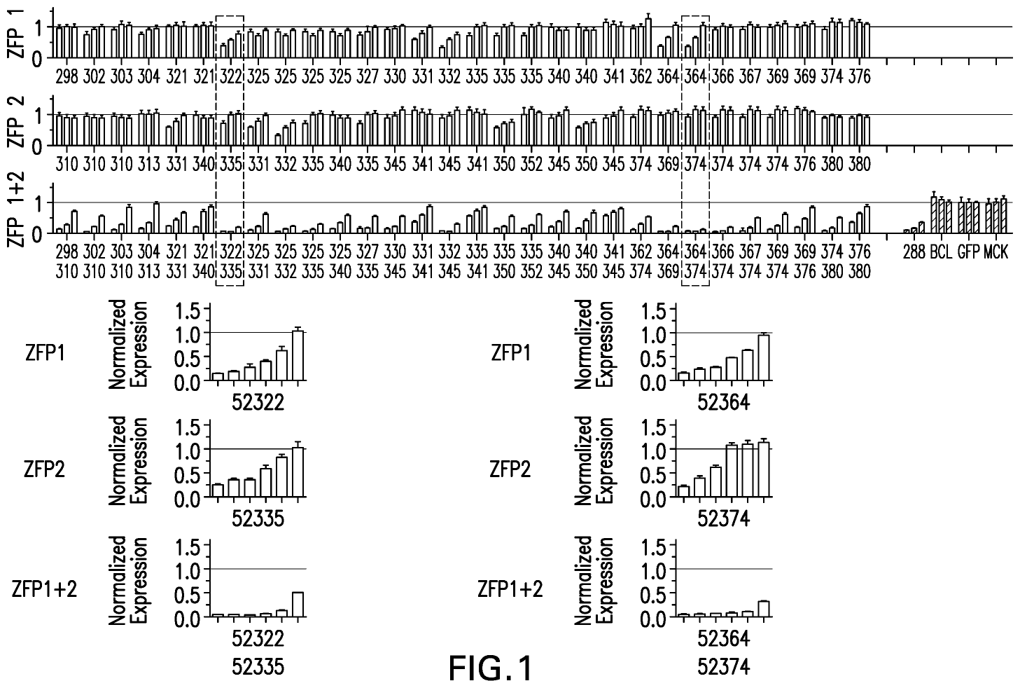

Based on the results of the initial screen (shown in the top 3 panels of FIG. 1), 4 ZFP-TFs 52322, 52335, 52364, 52374 and pairwise combinations thereof were selected for further study in which 6 different doses (300, 100, 30, 10, 3 and 1 ng) of the individual or combination ZFP-TFs were transfected into N2A cells and analyzed as described above. Synergy was also evaluated by comparing repression levels as between the individual ZFP-TFs and genetic modulators comprising a plurality of ZFP-TFs. The synergy score was calculated as the ratio of the expected normalized tau expression at the same nucleic acid dose for the strongest single ZFP-TF or modulator and the observed normalized tau expression when the ZFP or modulator combination is used.

As shown in the bottom panels of FIG. 1, genetic modulators comprising two ZFP-TF repressors repressed tau expression significantly more than the individual ZFP-TFs at the same dosages.

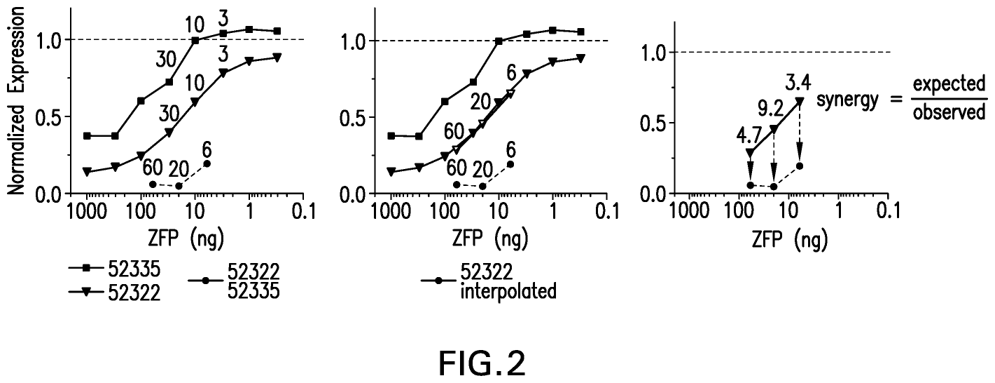

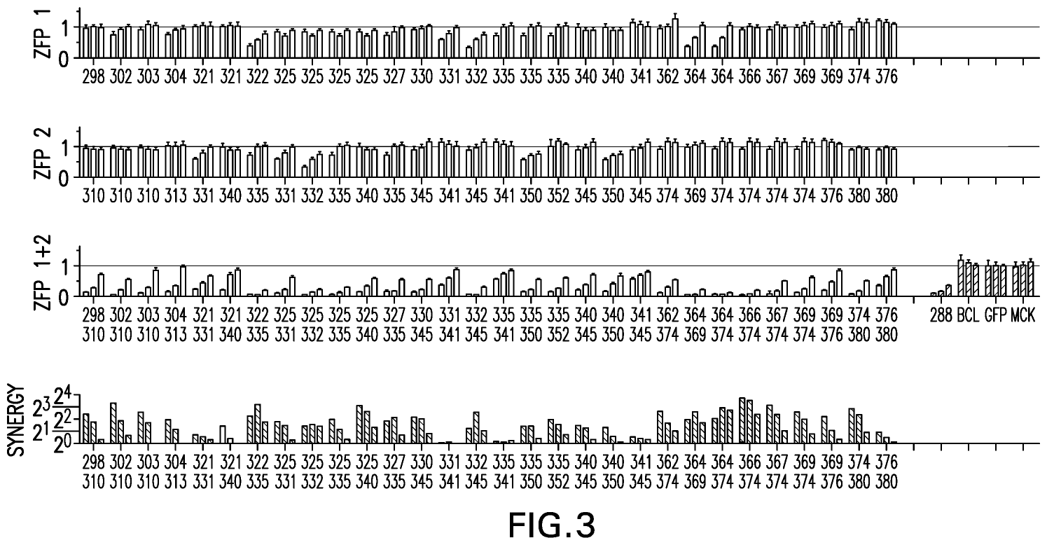

Further, as shown in FIGS. 2 and 3, repression of gene expression using ZFP-TFs comprising a plurality of ZFP-TFs provided surprising synergistic effects in the observed repression was 2- to 10- fold or more than the expected repression levels of the two ZFPs together.

Tables 1 and 2 shows exemplary designs used in various studies.

TABLE 1
Exemplary MAPT ZFP designs

| | Design | | | | | |
|---|---|---|---|---|---|---|
| SBS #, Target | F1 | F2 | F3 | F4 | F5 | F6 |
| SBS#52364 | RSDNLAR | DRSHLAR | QSGNLAR | QSNTRIM | N/A | N/A |
| cgACAGAAGGCGAG | (SEQ ID | (SEQ ID | (SEQ ID | (SEQ ID | | |
| gacagaagaggaca | NO: 7) | NO: 8) | NO: 9) | NO: 10) | | |
| (SEQ ID NO: 1) | | | | | | |
| SBS#52389 | ERGTLAR | TSANLSR | TSGNLTR | HRTSLTD | RSHSLLR | HPSARKR |
| ccGTTGCGCCTGAT | (SEQ ID | (SEQ ID | (SEQ ID | (SEQ ID | (SEQ ID | (SEQ ID |
| tGATGCCcagctcc | NO: 11) | NO: 12) | NO: 13) | NO: 14) | NO: 15) | NO: 16) |
| (SEQ ID NO: 2) | | | | | | |
| SBS#52322 | RSANLTR | DSSHLEL | DRSNLTR | DRSHLTR | DRSHLAR | N/A |
| gtGGCGGAGACTGA | (SEQ ID | (SEQ ID | (SEQ ID | (SEQ ID | (SEQ ID | |
| GAGcgcgcgcggcc | NO: 17) | NO: 18) | NO: 19) | NO: 20) | NO: 8) | |
| (SEQ ID NO: 3) | | | | | | |
| SBS# 57930 | DRSHLTR | LKQHLTR | RSAHLSR | TSGHLSR | QSGNLAR | QSGDLTR |
| cgGCAGAAGGTGG | (SEQ ID | (SEQ ID | (SEQ ID | (SEQ ID | (SEQ ID | (SEQ ID |
| GcGGTGGCggcggc | NO: 20) | NO: 37) | NO: 25) | NO: 26) | NO: 9) | NO: 35) |
| g (SEQ ID | | | | | | |
| NO: 44) | | | | | | |
| SBS# 57947 | RSAHLSR | TSGHLSR | QSGNLAR | QSGDLTR | DRSHLSR | DRSHLAR |
| gcGGCGGCgGCAG | (SEQ ID | (SEQ ID | (SEQ ID | (SEQ ID | (SEQ ID | (SEQ ID |
| AAGGTGGGcggtgg | NO: 25) | NO: 26) | NO: 9) | NO: 35) | NO: 38) | NO: 8) |
| C (SEQ ID | | | | | | |
| NO: 45) | | | | | | |
| SBS# 52335 | TSGHLSR | QSSDLSR | HRSTRNR | QSSDLSR | DRSHLAR | N/A |
| gcGGCGCTGCTGCT | (SEQ ID | (SEQ ID | (SEQ ID | (SEQ ID | (SEQ ID | |
| GGTgctggagctgg | NO: 26) | NO: 57) | NO: 58) | NO: 57) | NO: 8) | |
| (SEQ ID NO: 46) | | | | | | |
| SBS# 52374 | RSADLTR | RSDDLTR | QSSDLSR | QSTHRNA | N/A | N/A |
| tcAGAGCTGCGGCG | (SEQ ID | (SEQ ID | (SEQ ID | (SEQ ID | | |
| cttacctgataatc | NO: 34) | NO: 59) | NO: 57) | NO: 60) | | |
| (SEQ ID NO: 47) | | | | | | |
| SBS# 52244 | RSADLSR | DQSNLRA | RRSDLKR | RSDSLSR | DRSDRTK | N/A |
| atTCCGTGGCGCAC | (SEQ ID | (SEQ ID | (SEQ ID | (SEQ ID | (SEQ ID | |
| GCGcacccggcctc | NO: 61) | NO: 62) | NO: 63) | NO: 64) | NO: 65) | |
| (SEQ ID NO: 48) | | | | | | |
| SBS# 52345 | RSAHLSR | TSGHLSR | QSGNLAR | QSGDLTR | RSDDLTR | N/A |
| cgGCGGCAGAAGGT | (SEQ ID | (SEQ ID | (SEQ ID | (SEQ ID | (SEQ ID | |
| GGGcggtggcggcg | NO: 25) | NO: 26) | NO: 9) | NO: 35) | NO: 59) | |
| (SEQ ID NO: 49) | | | | | | |
| SBS# 52210 | YNKDLVK | DRSHLAR | QSGDLTR | LKDTLRR | LRHHLTR | DRSTLRQ |
| ccCCAAGTGCTACA | (SEQ ID | (SEQ ID | (SEQ ID | (SEQ ID | (SEQ ID | (SEQ ID |
| GGCTCCccgagtct | NO: 66) | NO: 8) | NO: 35) | NO: 67) | NO: 27) | NO: 68) |
| (SEQ ID NO: 50) | | | | | | |
| SBS# 52331 | QSSDLSR | QHGSLSR | QSSDLSR | WHSSLHQ | QSSDLSR | HRSTRNR |
| ctGCTGCTGGTGCT | (SEQ ID | (SEQ ID | (SEQ ID | (SEQ ID | (SEQ ID | (SEQ ID |
| GGAGCTggtgggtg | NO: 57) | NO: 69) | NO: 57) | NO: 70) | NO: 57) | NO: 58) |
| (SEQ ID NO: 51) | | | | | | |
| SBS# 52333 | RSADLTR | QSGDLTR | QSGDLTR | QSGTRLE | QSGNLAR | N/A |
| caGCACCAGCAGCA | (SEQ ID | (SEQ ID | (SEQ ID | (SEQ ID | (SEQ ID | |
| GCGccgccgccacc | NO: 34) | NO: 35) | NO: 35) | NO: 71) | NO: 9) | |
| (SEQ ID NO: 52) | | | | | | |
| SBS# 52313 | QSSDLSR | FRYYLKR | QSGNLAR | QRIDLTR | DRSTRTK | RRDTLLD |
| cgCCGGCCtCCAGA | (SEQ ID | (SEQ ID | (SEQ ID | (SEQ ID | (SEQ ID | (SEQ ID |
| ACGCGCTttctcgg | NO: 57) | NO: 72) | NO: 9) | NO: 73) | NO: 74) | NO: 75) |
| (SEQ ID NO: 53) | | | | | | |
| SBS# 52373 | QSGNLAR | LAYDRRK | RSDNLSA | RNNDRKT | RSADLTR | QSANRTK |
| ggTAAGCGCCGCAG | (SEQ ID | (SEQ ID | (SEQ ID | (SEQ ID | (SEQ ID | (SEQ ID |
| cTCTGAAatccagt | NO: 9) | NO: 76) | NO: 77) | NO: 78) | NO: 34) | NO: 79) |
| (SEQ ID NO: 54) | | | | | | |
| SBS# 52247 | DRSNLSR | LRQDLKR | TSGHLSR | YDYGRYT | DRSHLAR | N/A |
| cgGGCCGCGGTGCT | (SEQ ID | (SEQ ID | (SEQ ID | (SEQ ID | (SEQ ID | |
| GACcgccgcgcgca | NO: 39) | NO: 80) | NO: 26) | NO: 82) | NO: 8) | |
| (SEQ ID NO: 55) | | | | | | |
| SBS# 52365 | RSDNLSV | ASWTLTQ | QNAHRKT | TSSNRKT | TSGNLTR | HRTSLTD |
| taCCTGATAATCGA | (SEQ ID | (SEQ ID | (SEQ ID | (SEQ ID | (SEQ ID | (SEQ ID |
| CAGAAGgcgaggac | NO: 82) | NO: 83) | NO: 84) | NO: 22) | NO: 13) | NO: 14) |
| (SEQ ID NO: 56) | | | | | | |
| SBS# 52366 | RSDNLSE | TSSNRKT | TSGNLTR | DRSALAR | RNSDRTK | N/A |
| ctTCTGTCGATTAT | (SEQ ID | (SEQ ID | (SEQ ID | (SEQ ID | (SEQ ID | |
| CAGgtaagcgccgc | NO: 21) | NO: 22) | NO: 13) | NO: 23) | NO: 24) | |
| (SEQ ID NO: 4) | | | | | | |
| SBS# 57880 | DSSHLEL | DRSNLTR | DRSHLTR | DRSHLAR | RSAHLSR | TSGHLSR |
| ctGGTGGGtGGCGG | (SEQ ID | (SEQ ID | (SEQ ID | (SEQ ID | (SEQ ID | (SEQ ID |
| AGACTGAgagcgcg | NO: 18) | NO: 19) | NO: 20) | NO: 8) | NO: 25) | NO: 26) |
| (SEQ ID NO: 5) | | | | | | |
| SBS# 57890 | LRHHLTR | RRFTLSK | RSDVLSE | KHSTRRV | RSDVLSE | RLYTLHK |
| tgGTGCTGGAGCT | (SEQ ID | (SEQ ID | (SEQ ID | (SEQ ID | (SEQ ID | (SEQ ID |
| GGTGGGTggcggag | NO: 27) | NO: 28) | NO: 29) | NO: 30) | NO: 29) | NO: 30) |
| a (SEQ ID | | | | | | |
| NO: 6) | | | | | | |
| SBS# 52288 | RSADLTR | QSGDLTR | RSDHLSE | RSAHLSR | N/A | N/A |
| agGGGCGGGCAGCG | (SEQ ID | (SEQ ID | (SEQ ID | (SEQ ID | | |
| aggcctgggcgggc | NO: 34) | NO: 35) | NO: 36) | NO: 25) | | |
| (SEQ ID NO: 33) | | | | | | |
| SBS# 65976 | DRSNLSR | LRQNLIM | TSANLTV | RSDHLSR | QSGNLAR | QRNDRKS |
| ctCCAGAAGGGGAT | (SEQ ID | (SEQ ID | (SEQ ID | (SEQ ID | (SEQ ID | (SEQ ID |
| CATGACctcctcac | NO: 39) | NO: 40) | NO: 41) | NO: 42) | NO: 9) | NO: 43) |
| (SEQ ID NO: 46) | | | | | | |
| 65976 Phos | Qm5 | none | Qm5 | none | Qm5 | none |
| contact | | | | | | |
| mutants | | | | | | |

TABLE 2
Exemplary optimized ZFP-TF designs

| | Design | | | | | |
|---|---|---|---|---|---|---|
| SBS #, Target | F1 | F2 | F3 | F4 | F5 | F6 |
| SBS# 57930 | DRSHLTR | LKQHLTR | RSAHLSR | TSGHLSR | QSGNLAR | QSGDLTR |
| cgGCAGAAGGTGG | (SEQ ID | (SEQ ID | (SEQ ID | (SEQ ID | (SEQ ID | (SEQ ID |
| GcGGTGGCggcggc | NO: 20) | NO: 37) | NO: 25) | NO: 26) | NO: 9) | NO: 35) |
| g (SEQ ID | | | | | | |
| NO: 44) | | | | | | |
| [Parent] | | | | | | |
| SBS# 65918 | DRSHLTR | LKQHLTR | RSAHLSR | TSGHLSR | QSGNLAR | QSGDLTR |
| cgGCAGAAGGTGG | (SEQ ID | (SEQ ID | (SEQ ID | (SEQ ID | (SEQ ID | (SEQ ID |
| GcGGTGGCggcggc | NO: 20) | NO: 37) | NO: 25) | NO: 26) | NO: 9) | NO: 35) |
| g (SEQ ID | | | | | | |
| NO: 44) | | | | | | |
| 65918 Phos | Qm5 | none | none | none | Qm5 | none |
| contact | | | | | | |
| mutants | | | | | | |
| SBS# 65920 | DRSHLTR | LKQHLTR | RSAHLSR | TSGHLSR | QSGNLAR | QSGDLTR |
| cgGCAGAAGGTGG | (SEQ ID | (SEQ ID | (SEQ ID | (SEQ ID | (SEQ ID | (SEQ ID |
| GcGGTGGCggcggc | NO: 20) | NO: 37) | NO: 25) | NO: 26) | NO: 9) | NO: 35) |
| g (SEQ ID | | | | | | |
| NO: 44) | | | | | | |
| 65920 Phos | Qm5 | none | Qm5 | none | Qm5 | none |
| contact | | | | | | |
| mutants | | | | | | |
| SBS# 57890 | LRHHLTR | RRFTLSK | RSDVLSE | KHSTRRV | RSDVLSE | RLYTLHK |
| tgGTGCTGGAGCT | (SEQ ID | (SEQ ID | (SEQ ID | (SEQ ID | (SEQ ID | (SEQ ID |
| GGTGGGTggcggag | NO: 27) | NO: 28) | NO: 29) | NO: 30) | NO: 29) | NO: 30) |
| a (SEQ ID | | | | | | |
| NO: 6) | | | | | | |
| [Parent] | | | | | | |
| SBS# 65894 | LRHHLTR | RRFTLSK | RSDVLSE | KHSTRRV | RSDVLSE | RLYTLHK |
| tgGTGCTGGAGCT | (SEQ ID | (SEQ ID | (SEQ ID | (SEQ ID | (SEQ ID | (SEQ ID |
| GGTGGGTggcggag | NO: 27) | NO: 28) | NO: 29) | NO: 30) | NO: 29) | NO: 30) |
| a (SEQ ID | | | | | | |
| NO: 6) | | | | | | |
| 65894 Phos | Qm5 | none | none | none | Qm5 | none |
| contact | | | | | | |
| mutants | | | | | | |
| SBS# 57947 | RSAHLSR | TSGHLSR | QSGNLAR | QSGDLTR | DRSHLSR | DRSHLAR |
| gcGGCGGCgGCAG | (SEQ ID | (SEQ ID | (SEQ ID | (SEQ ID | (SEQ ID | (SEQ ID |
| AAGGTGGGcggtgg | NO: 25) | NO: 26) | NO: 9) | NO: 35) | NO: 38) | NO: 8) |
| c (SEQ ID | | | | | | |
| NO: 45) | | | | | | |
| [parent] | | | | | | |
| SBS# 65968 | RSAHLSR | TSGHLSR | QSGNLAR | QSGDLTR | DRSHLSR | DRSHLAR |
| gcGGCGGCgGCAG | (SEQ ID | (SEQ ID | (SEQ ID | (SEQ ID | (SEQ ID | (SEQ ID |
| AAGGTGGGcggtgg | NO: 25) | NO: 26) | NO: 9) | NO: 35) | NO: 38) | NO: 8) |
| c (SEQ ID | | | | | | |
| NO: 45) | | | | | | |
| 65968 Phos | Qm5 | none | Qm5 | none | Qm5 | none |
| contact | | | | | | |
| mutants | | | | | | |
| SBS#57880 | DSSHLEL | DRSNLTR | DRSHLTR | DRSHLAR | RSAHLSR | TSGHLSR |
| ctGGTGGGtGGCGG | (SEQ ID | (SEQ ID | (SEQ ID | (SEQ ID | (SEQ ID | (SEQ ID |
| AGACTGAgagcgcg | NO: 18) | NO: 19) | NO: 20) | NO: 8) | NO: 25) | NO: 26) |
| (SEQ ID NO: 5) | | | | | | |
| [Parent] | | | | | | |
| SBS#65888 | DSSHLEL | DRSNLTR | DRSHLTR | DRSHLAR | RSAHLSR | TSGHLSR |
| ctGGTGGGtGGCGG | (SEQ ID | (SEQ ID | (SEQ ID | (SEQ ID | (SEQ ID | (SEQ ID |
| AGACTGAgagcgcg | NO: 18) | NO: 19) | NO: 20) | NO: 8) | NO: 25) | NO: 26) |
| (SEQ ID NO: 5) | | | | | | |
| 65888 Phos | Qm5 | none | Qm5 | none | Qm5 | none |
| contact | | | | | | |
| mutants | | | | | | |
| SBS#65887 | DSSHLEL | DRSNLTR | DRSHLTR | DRSHLAR | RSAHLSR | TSGHLSR |
| ctGGTGGGtGGCGG | (SEQ ID | (SEQ ID | (SEQ ID | (SEQ ID | (SEQ ID | (SEQ ID |
| AGACTGAgagcgcg | NO: 18) | NO: 19) | NO: 20) | NO: 8) | NO: 25) | NO: 26) |
| (SEQ ID NO: 5) | | | | | | |
| 65887 Phos | none | none | Qm5 | none | Qm5 | none |
| contact | | | | | | |
| mutants | | | | | | |
| SBS#52389 | ERGTLAR | TSANLSR | TSGNLTR | HRTSLTD | RSHSLLR | HPSARKR |
| ccGTTGCGCCTGAT | (SEQ ID | (SEQ ID | (SEQ ID | (SEQ ID | (SEQ ID | (SEQ ID |
| tGATGCCcagctcc | NO: 11) | NO: 12) | NO: 13) | NO: 14) | NO: 15) | NO: 16) |
| (SEQ ID NO: 2) | | | | | | |
| [Parent] | | | | | | |
| SBS#65860 | ERGTLAR | TSANLSR | TSGNLTR | HRTSLTD | RSHSLLR | HPSARKR |
| ccGTTGCGCCTGAT | (SEQ ID | (SEQ ID | (SEQ ID | (SEQ ID | (SEQ ID | (SEQ ID |
| tGATGCCcagctcc | NO: 11) | NO: 12) | NO: 13) | NO: 14) | NO: 15) | NO: 16) |
| (SEQ ID NO: 2) | | | | | | |
| 65860 Phos | none | none | none | none | Qm5 | none |
| contact | | | | | | |
| mutants | | | | | | |

Genetic modulators comprising two artificial transcription factors described above were also evaluated for synergistic effects based on (1) distance between repression (KRAB) domains (in nucleotides); (2) distance of the target site bound from the transcription start site (TSS); (3) distance of the target sites as between the two ZFP-TFs; and (4) strand to which the individual ZFP-TFs bind as follows as follows. The synergy score was calculated as the ratio of the expected normalized tau expression at the same nucleic acid dose for the strongest single ZFP-TF or modulator and the observed normalized tau expression when the ZFP or modulator combination is used. Synergy was evaluated at 30 ng dose shown for 368 pairs (made from combinations 43 singles).

As shown in FIG. 4, synergistic effects (repression) were readily achieved using two ZFP-TFs at distances up to 600 base pairs between the two target sites or repression domains; with ZFP-TFs having a central distance between their two target sites within 200 base pairs (3′ or 5′) of the TSS; and regardless of which strand of the target sequence bound the ZFP-TFs.

Subsequently, studies were conducted to further evaluate genetic repressors including at least two artificial transcription factors that act synergistically. A panel of active individual ZFP-TFs was identified and tested in a full matrix of all paired combinations, as well as testing of all 6 ZFP-TFs delivered together.

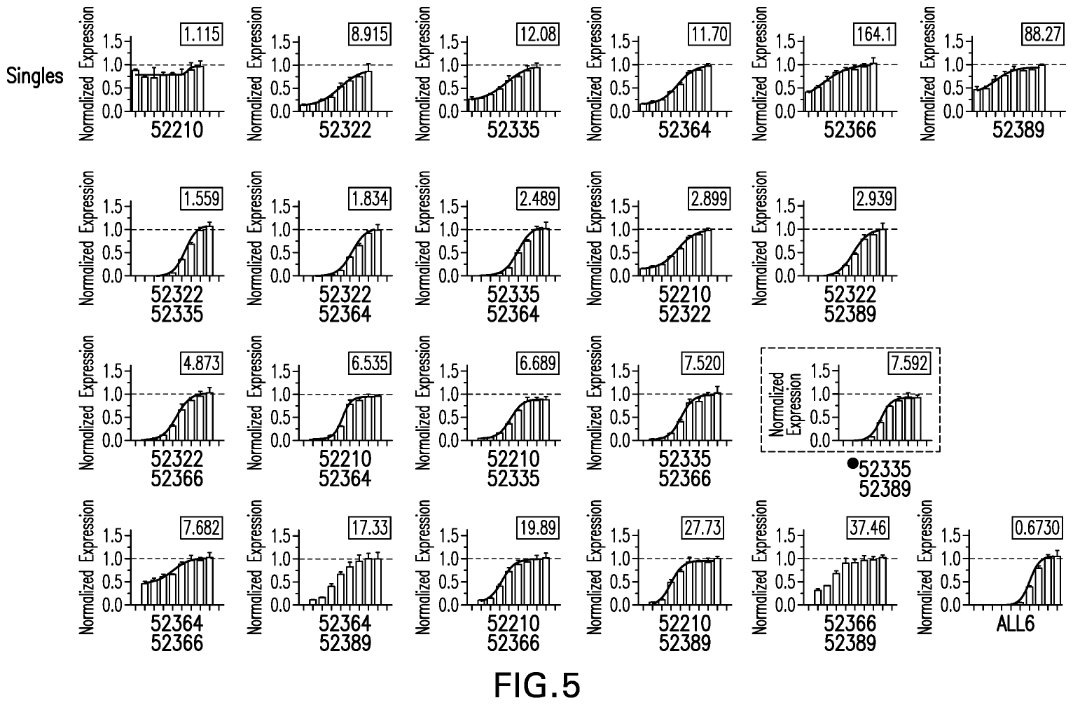

Results for exemplary identified singles, pairs, and multiple combinations are shown in FIG. 5 and confirm synergistic effects are readily achieved by combining two or more ZFP-TFs in almost any combination. An experiment where all 6 ZFP-TFs were co-delivered resulted in the greatest levels of tau reduction and lowest EC50, approximately 3× lower than the most potent ZFP pair 52322-52335.

### B. Mouse Prion (Prnp)

ZFP-TFs targeted to the mouse Prnp gene were also screened for synergistic effects, essentially as described above. Briefly, 3 different doses (200, 60, 20 ng for individual ZFP-TFs and 100, 30 and 10 ng for paired combinations) of mRNA encoding 32 different individual ZFP-TFs and 130 different pairwise combinations of these ZFP-TFs were transfected into Neuro2A cells. After 24 hours, total RNA was extracted and the expression of Prnp and two reference genes (ATP5b, EIF4A) was monitored using real-time RT-qPCR. Synergy was calculated as the ratio of the expression level obtained with the stronger ZFP when tested at 2× of its dose in the combination to that obtained with the ZFP combination.

Results showing synergistic effects for the 130 ZFP-TF combinations tested are shown below in Table A.

TABLE A
Overall Synergy Mouse Prnp

| Fold Synergy | Percentage of Combinations |
|---|---|
| >1X | 76.2% |
| >2X | 43.1% |
| >4X | 10.0% |
| >6X | 3.8% |
| >8X | 0.8% |

Thus, a synergistic effect (as compared to the single TFs) was observed in more than 75% of the combinations tested, with a greater than 2-fold synergistic effect observed in over 40% of tested combinations.

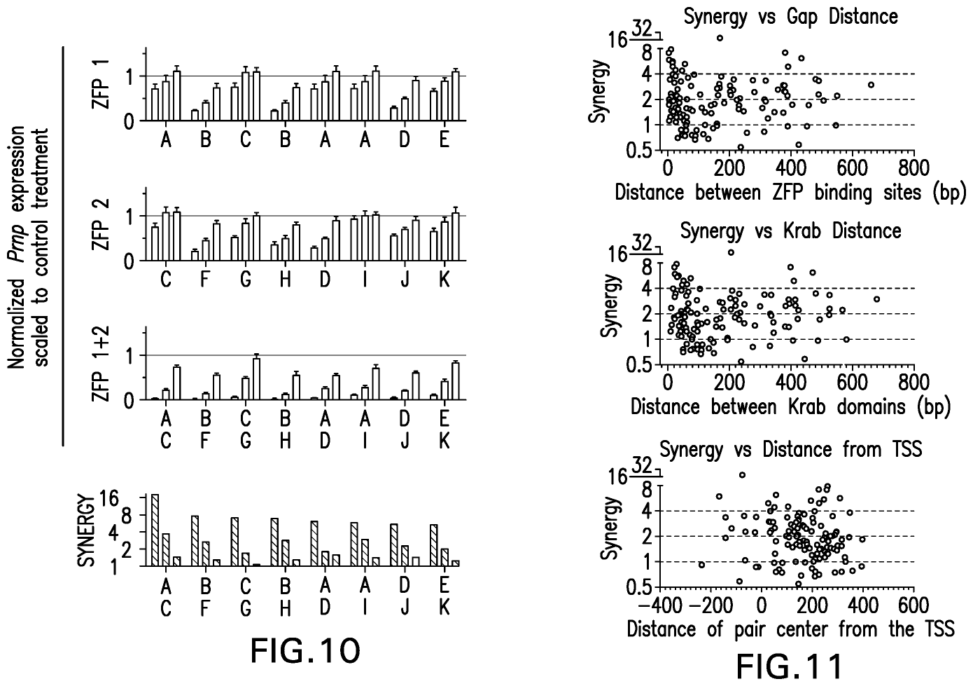

In addition, FIG. 10 graphically shows synergistic effects of 8 exemplary ZFT-TFs combinations as compared to the ZFP-TFs individually. The Exemplary ZFP-TFs shown are designated A to K.

The 130 combinations of mouse prion ZFP-TFs were also evaluated for synergistic effects based on (1) distance between repression (KRAB) domains (in nucleotides); (2) distance of the target site bound from the transcription start site (TSS); and (3) distance of the target sites as between the two ZFP-TFs. Synergy was calculated as described above.

As shown in FIG. 11, synergistic effects (repression) were readily achieved using two ZFP-TFs at distances up to 600 base pairs between the two target sites or between repression domains; and with ZFP-TFs having a central distance between their two target sites within 600 base pairs (3′ or 5′) of the TSS.

### C. Human Prion (PRNP)

ZFP-TFs targeted to human PRNP gene were also screened for synergistic effects, essentially as described above. Briefly, 3 different doses (200, 60, 20 ng for individual ZFP-TFs and 100, 30 and 10 ng for paired combinations) of mRNA encoding 32 different individual ZFP-TFs and 130 different pairwise combinations of these ZFP-TFs were transfected into SK-N-MC cells. After 24 hours, total RNA was extracted and the expression of PRNP and two reference genes (ATP5b, EIF4A) was monitored using real-time RT-qPCR. Synergy was calculated as the ratio of the expression level obtained with the stronger ZFP when tested at 2× of its dose in the combination to the expression level obtained with the ZFP combination.

Results showing synergistic effects for the 130 ZFP-TF combinations tested are shown below in Table B.

TABLE B
Overall Synergy Human PRNP

| Fold Synergy | Percentage of Combinations |
|---|---|
| >1X | 66.2% |
| >2X | 23.8% |
| >4X | 7.7% |
| >6X | 5.4% |
| >8X | 0.0% |

Thus, a synergistic effect (as compared to the single TFs) was observed in more than 66% of the combinations tested, with a greater than 2-fold synergistic effect observed in over 23% of tested combinations.

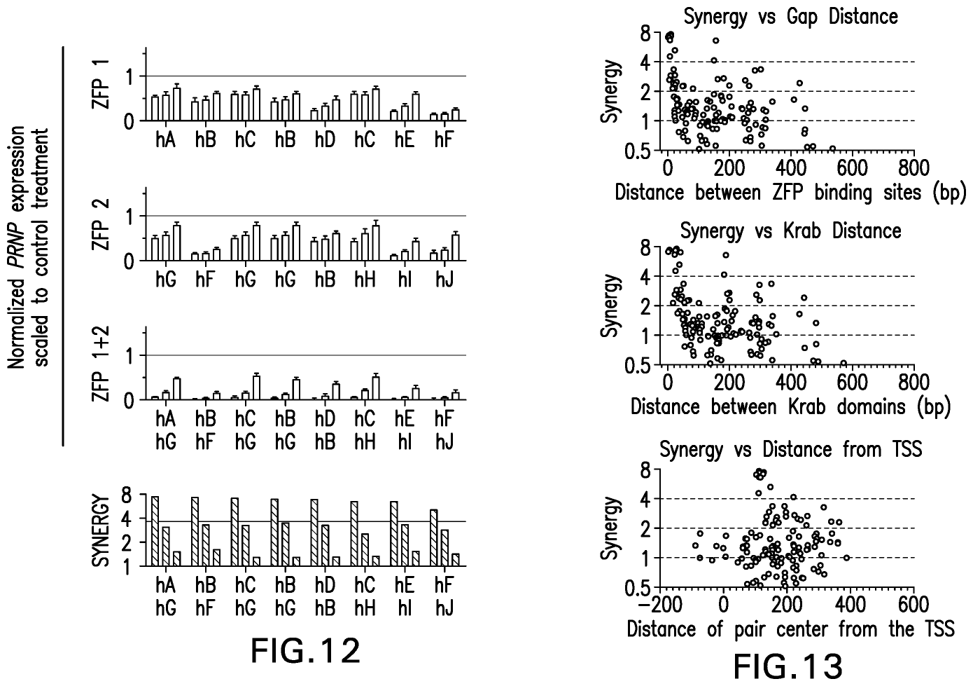

In addition, FIG. 12 graphically shows synergistic effects of 8 exemplary ZFT-TFs combinations as compared to the ZFP-TFs individually. The Exemplary ZFP-TFs shown are designated hA to hJ.

The 130 combinations of human prion ZFP-TFs were also evaluated for synergistic effects based on (1) distance between repression (KRAB) domains (in nucleotides); (2) distance of the target site bound from the transcription start site (TSS); and (3) distance between the target sites of the two ZFP-TFs. Synergy was calculated as described above.

As shown in FIG. 13, synergistic effects (repression) were readily achieved using two ZFP-TFs at distances up to 600 base pairs between the two target sites or between repression domains; and with ZFP-TFs having a central distance between their two target sites within 600 base pairs (3′ or 5′) of the TSS.

### D. Human α-Synuclein (SNCA)

ZFP-TFs targeted to human SNCA were also screened for synergistic effects, essentially as described above. Briefly, 3 different doses (200, 60, 20 ng for individual ZFP-TFs and 100, 30 and 10 ng for paired combinations) of mRNA encoding 30 different individual ZFP-TFs and 132 different pairwise combinations of these ZFP-TFs were transfected into SK-N-MC cells. After 24 hours, total RNA was extracted and the expression of SNCA and two reference genes (ATP5b, EIF4A) was monitored using real-time RT-qPCR. Synergy was calculated as the ratio of expression level obtained with the stronger ZFP when tested at 2× of its dose in the combination to that obtained with the ZFP combination.

Results showing synergistic effects for the 132 ZFP-TF combinations tested are shown below in Table C.

TABLE C
Overall Synergy Human SNCA

| Fold Synergy | Percentage of Combinations |
|---|---|
| >1X | 65.9% |
| >2X | 17.4% |
| >4X | 1.5% |
| >6X | 0.0% |
| >8X | 0.0% |

Thus, a synergistic effect (as compared to the single TFs) was observed in approximately 66% of the combinations tested, with a greater than 2-fold synergistic effect observed in over 17% of tested combinations.

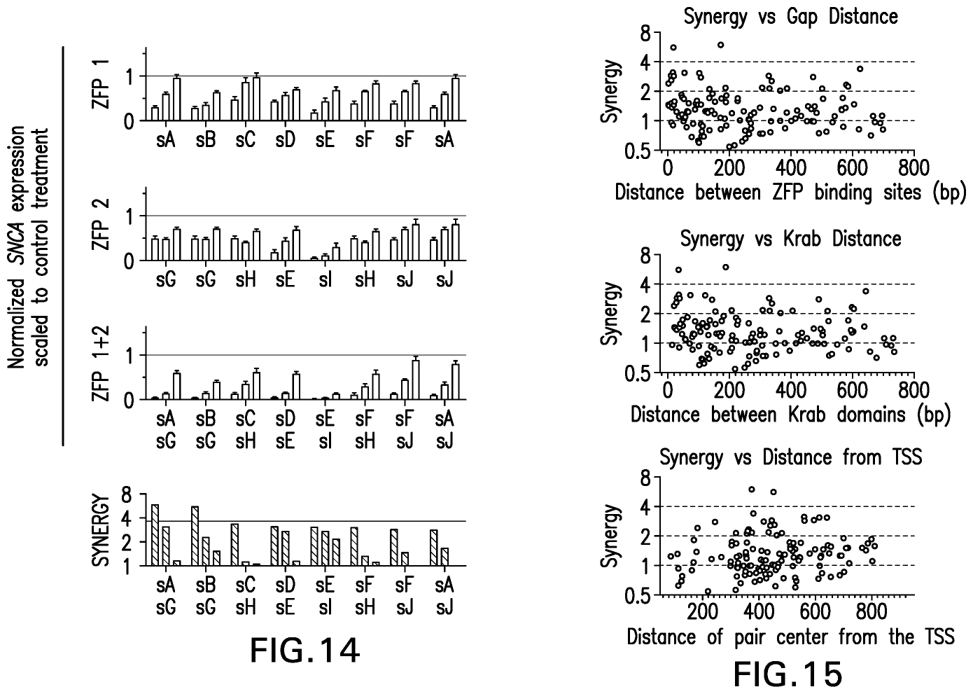

In addition, FIG. 14 graphically shows synergistic effects of 8 exemplary ZFT-TFs combinations as compared to the ZFP-TFs individually. The exemplary ZFP-TFs shown are designated sA to sJ.

The 132 combinations of human a-synuclein ZFP-TFs were also evaluated for synergistic effects based on (1) distance between repression (KRAB) domains (in nucleotides); (2) distance of the target site bound from the transcription start site (TSS); and (3) distance between the target sites of the two ZFP-TFs. Synergy was calculated as described above.

As shown in FIG. 15, synergistic effects (repression) were readily achieved using two ZFP-TFs at distances up to 600-800 base pairs between the two target sites or between repression domains; and with ZFP-TFs having a central distance between their two target sites within 600-800 base pairs (3′ or 5′) of the TSS.

### Example 2: Off-Target Effects

Off-target effects were also analyzed as follows. First, the pair 52335 and 52389 identified in Example 1 was used in global microarray profiling. In brief, about 300 ng of each ZFP-TF encoding mRNA was transfected into 150k Neuro2A cells in biological quadruplicate either individually or in combination. After about 24 hours, total RNA was extracted and processed via the manufacturer's protocol (Affymetrix Genechip MTA1.0). Robust Multi-array Average (RMA) was used to normalize raw signals from each probe set. Analysis was performed using Transcriptome Analysis Console 3.0 (Affymetrix) with the “Gene Level Differential Expression Analysis” option. ZFP-transfected samples were compared to samples that had been treated with an irrelevant ZFP-TF (that does not bind to MAPT target site). Change calls were reported for transcripts (probe sets) with a >2 fold difference in mean signal relative to control, and a P-value <0.05 (one-way ANOVA analysis, unpaired T-test for each probeset).

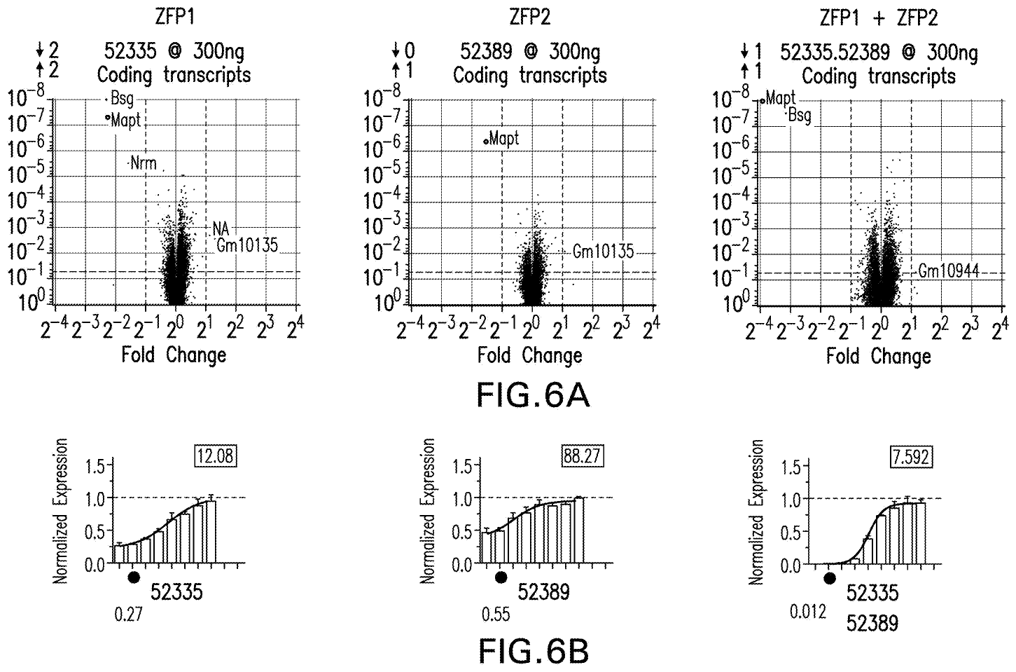

As shown in FIG. 6A, individual ZFP-TF 52335 repressed 2 non-target genes and activated one gene; individual ZFP-TF 52389 activated one gene; while the combination of ZFP-TFs and 52335 and 52389 activated one off-target site and repressed one off-target site. In addition, as shown in FIG. 6B, the genetic modulator comprising two ZFP-TF repressors repressed tau levels more than the individual repressors (0.012× wild-type levels), demonstrating that a substantial increase in on-target repression is achievable without increasing the number of off-targets.

### Example 3: Delivery

Multi-cistronic delivery and codon-diversified repression domains were also analyzed as follows. mRNA was generated encoding either a single ZFP-TF (unlinked) or as multi-cistronic (linked) with one mRNA carrying multiple artificial transcription factors (separated by self-cleaving peptide sequences, T2A and P2A). In addition, the ZFP-TFs comprised wild-type or codon-diversified variants of the Kox repression domain (designated nKox, mKox, and cKox for the N-terminal, Middle, or C-terminal position within the linked architecture, respectively) to avoid repetitive sequences in the delivery vectors.

The mRNAs were transfected into Neuro2A cells at the following doses: unlinked mRNA was transfected at doses of about 300, 100, 30, 10, 3, 1, 0.3 and 01 ng mRNA and bi-cistronic mRNA was transfected at doses of about 600, 200, 60, 20, 6, 2, 0.6, and 0.1 ng of mRNA. Tau gene expression levels were measured after about 24 hours.

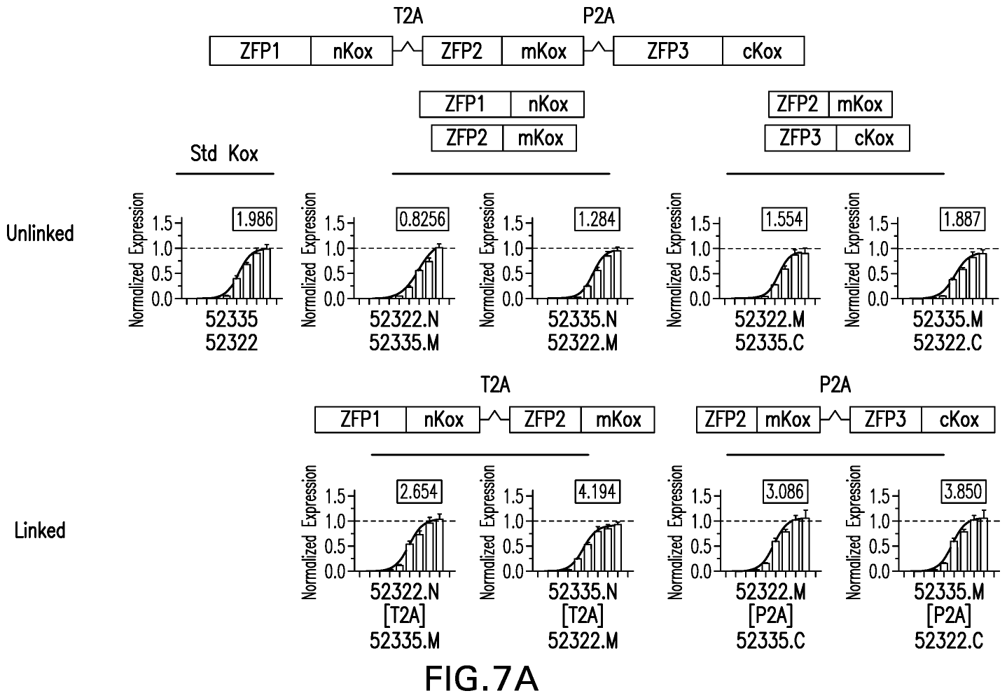

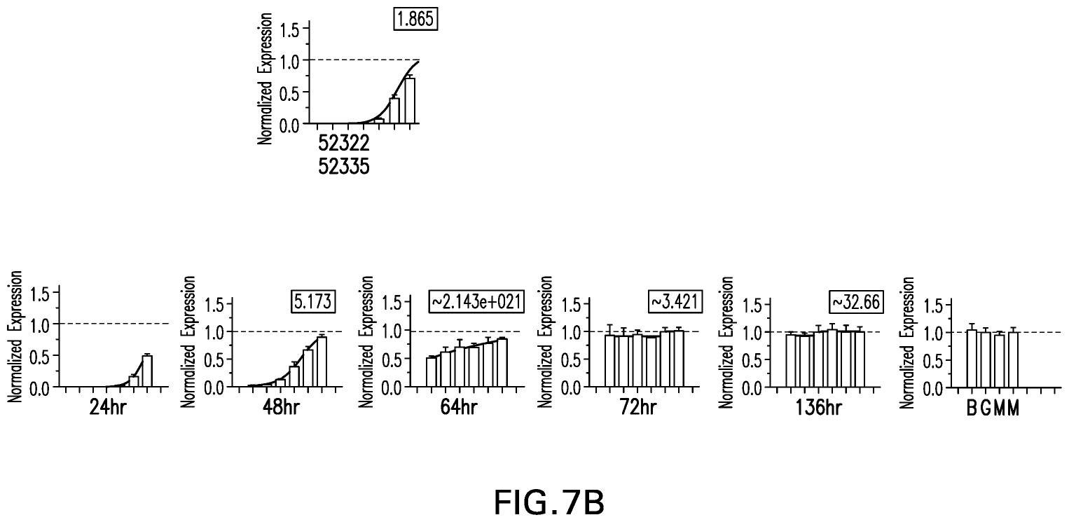

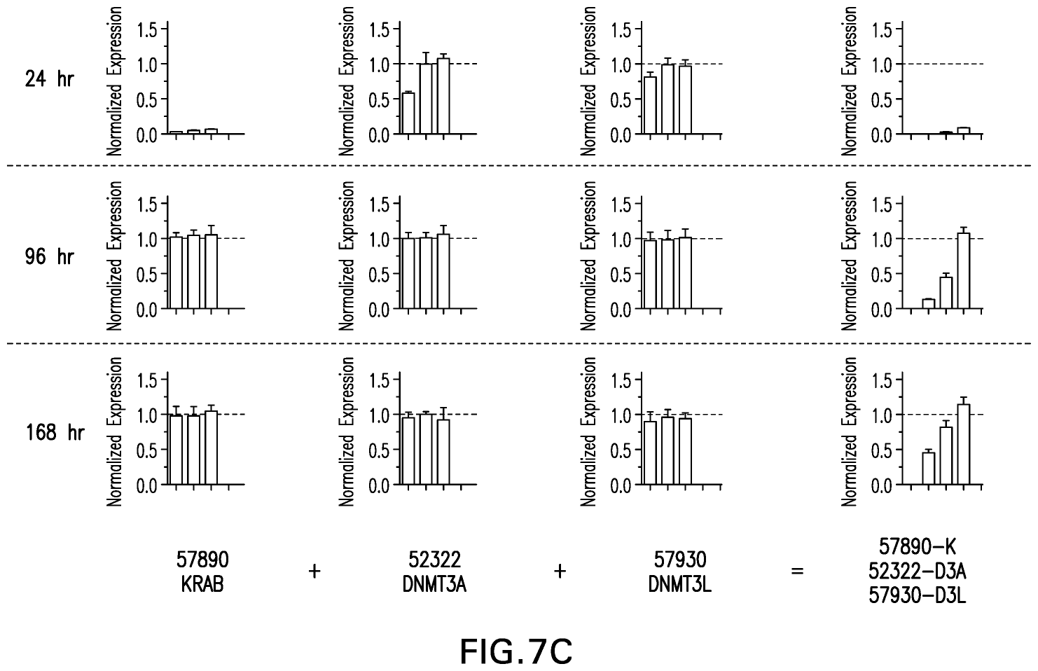

As shown in FIG. 7A, gene expression was effectively repressed by both linked and unlinked constructs to a similar magnitude and EC50 regardless of the Kox domain variant or cistronic architecture.

AAV vectors comprising polynucleotides encoding genetic repressors are also generated. The delivery vehicles carry either a single ZFP-TF (unlinked) or are multi-cistronic (linked) in that one AAV vector carries two or more artificial transcription factors of the genetic modulator. As with mRNA, both single and multi-cistronic AAV vectors repressed tau expression, indicating the single AAV vector encoding all the components of the genetic repressors described herein can be used.

The kinetics of gene modulation (repression) was also tested over time. In particular, gene expression (tau) levels were evaluated about 24, 48, 64, 72 and 136 hours after mRNA transfection. As shown in FIG. 7B, repression was not detectable at about 72 hours or longer post-transfection.

Furthermore, the effects of additional functional domains (DMNT) were also evaluated over various time points. Three ZFP fusion proteins were generated as follows: ZFP 57890 operably linked to a KRAB repression domain (57890-K); ZFP 52322 operably linked to a DNMT3A functional domain (52322-D3A) and ZFP 57930 operably linked to a DNMT3L functional domain and transfected into N2A cells individually at doses of about 900, 300 or 100 ng or together at doses of about 300, 100 or 30 ng. Cells were harvested after about 24, 96 or 168 hours and gene expression levels evaluated.

As shown in FIG. 7C, the triple transfection gave robust levels of repression up to about 168 hours after transfection, whereas delivery of any single ZFP modulator was unable to repress tau expression beyond 24 hours.

### Example 4: In Vivo Non-Human Primate Studies

Genetic repressors as described herein were tested in cynomolgus monkeys (M. fascicularis) to observe repression of tau expression in a primate (non-human primate (NHP) model). Cynomolgus monkeys were housed in stainless steel cages equipped with an automatic watering system. The study complied with all applicable sections of the Final Rules of the Animal Welfare Act regulations (Code of Federal Regulations, Title 9) and the Guide for the Care and Use of Laboratory Animals, Institute of Laboratory Animal Resources, Commission on Life Sciences, National Research Council, 8th edition.

The genetic repressors were cloned into an AAV vector (AAV2/9, or variants thereof) with the SYN1 promoter or CMV promoter, essentially as described in U.S. Publication No. 20180153921. AAV vectors used included: a vector with a SYN1 promoter driving expression of genetic modulators as described herein comprising 65918 and 57890 (SYN918-890) and a vector with a CMV promoter driving expression of a genetic modulator comprising 65918 and 57890 (CMV918-890).

NHP subjects were treated as shown in the following Table:

TABLE 4

| | | rAAV |
|---|---|---|
| Cohort | AAV-promoter ZFP | vg/kg |
| NHP01 | Vehicle only | 0 |
| NHP02 | Vehicle only | 0 |
| NHP03 | Vehicle only | 0 |
| NHP04 | Synapsin-65918 and 57890 | 6E11 |
| NHP05 | Synapsin-65918 and 57890 | 6E11 |
| NHP06 | Synapsin-65918 and 57890 | 6E11 |
| NHP07 | CMV-65918 and 57890 | 6E11 |
| NHP08 | CMV-65918 and 57890 | 6E11 |

In the experiment, AAV9 vectors comprising a hSYN1 or CMV driven ZFP TF are delivered at about 6E11 vg/hemisphere to the left and about 6E11 vg/hemisphere to the right hemisphere. Animals received a single dose of test article in a volume of about 60 μL in the left and a single dose of about 60 μL in the right hemisphere. For all test articles, the dose concentration was about 1E13 vg/mL.

After 28 days, the animals were sacrificed, and the brains were removed and placed in a coronal brain matrix in ice-cold PBS. Brains were sliced at a 3 mm coronal slice thickness (divided into approximately 17 slices). Some brain slices (right and left hemisphere) were stored in 10% neutral-buffered formalin for histopathology and in situ hybridization analyses. All other brain slices (right and left hemisphere) were placed in RNAlater (Qiagen) and refrigerated for approximately 24 hours, after which 2-3 mm diameter punches were collected according to a predefined brain template. Punches were processed for qRT-PCR and biodistribution analysis. Additionally, CSF was collected for tau protein analysis.

Slices comprising the hippocampus and entorhinal cortex regions were used to analyze mRNA expression levels of tau, ZFP, glial and neuronal cell markers, and housekeeping genes via qRT-PCR. The results show that the ZFP-TFs were delivered by AAV to the hippocampal region leading to reduction in tau expression.

Subjects were necropsied at 28 days post infusion, the brains were removed and sections into 3 mm coronal blocks along the rostral-caudal access, and punch biopsies were collected from each block for several brain regions, including the hippocampus and entorhinal cortex. qRT-PCR was used to evaluate tau expression, housekeeping gene (ATP5b, EIF4a2, and GAPDH), and ZFP expression levels in 74 punches form different brain sections.

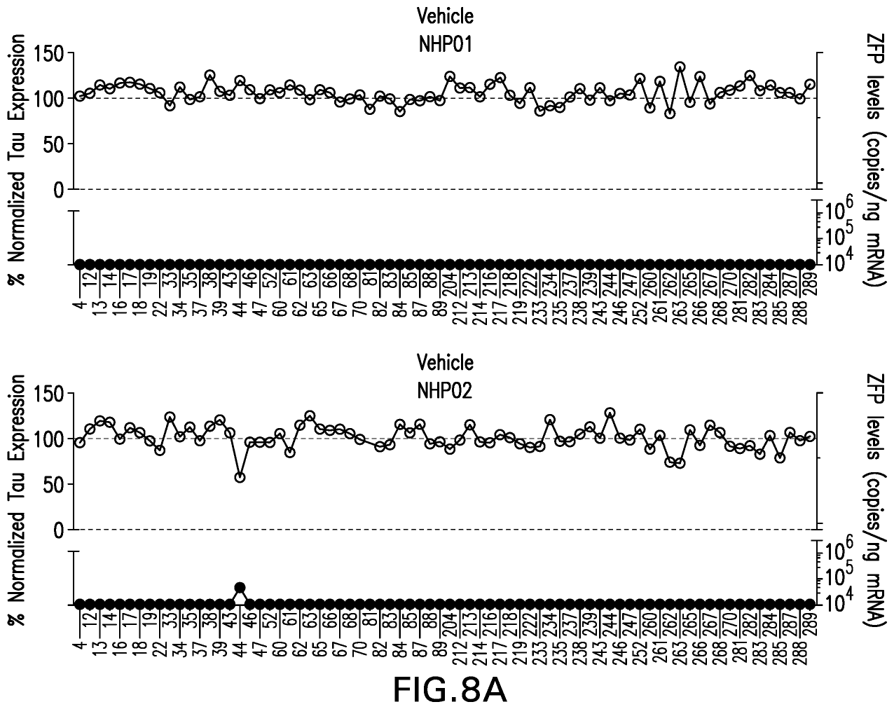

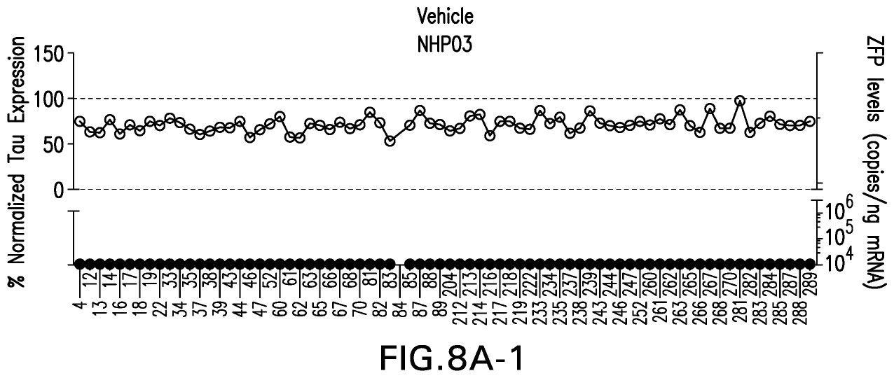

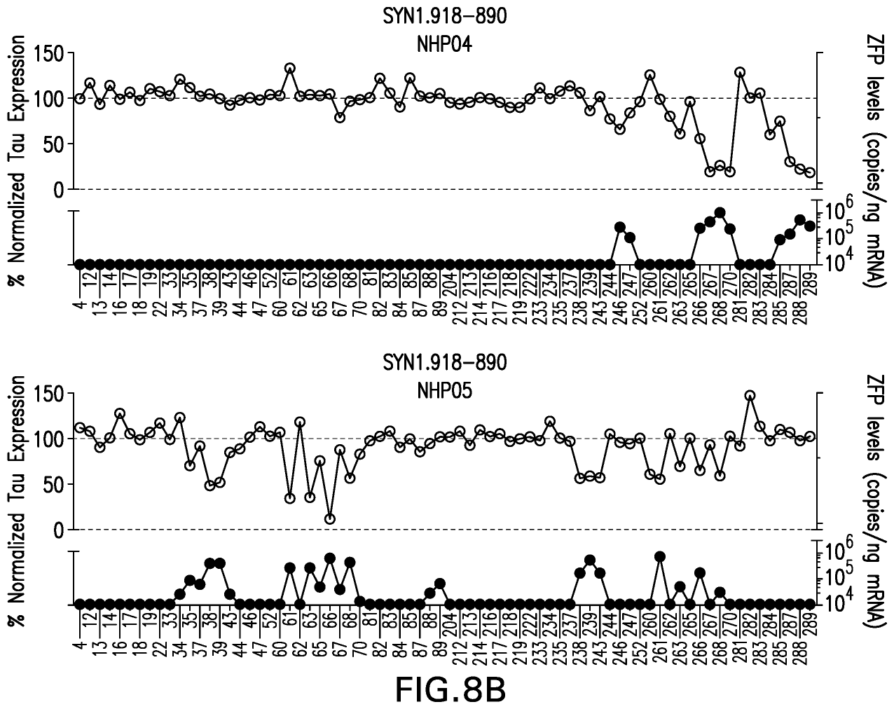

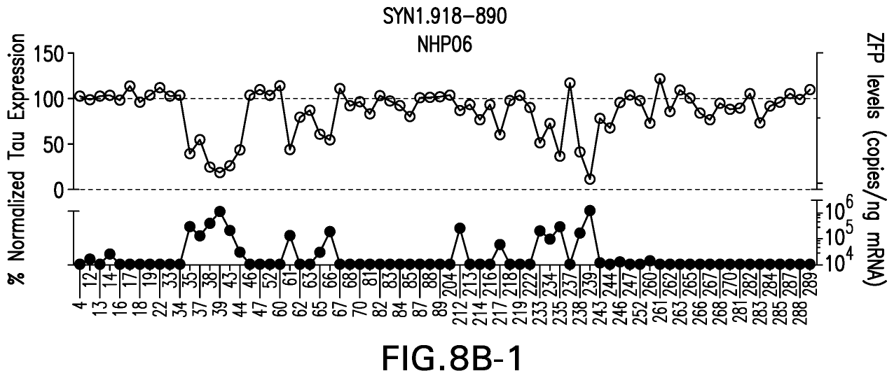

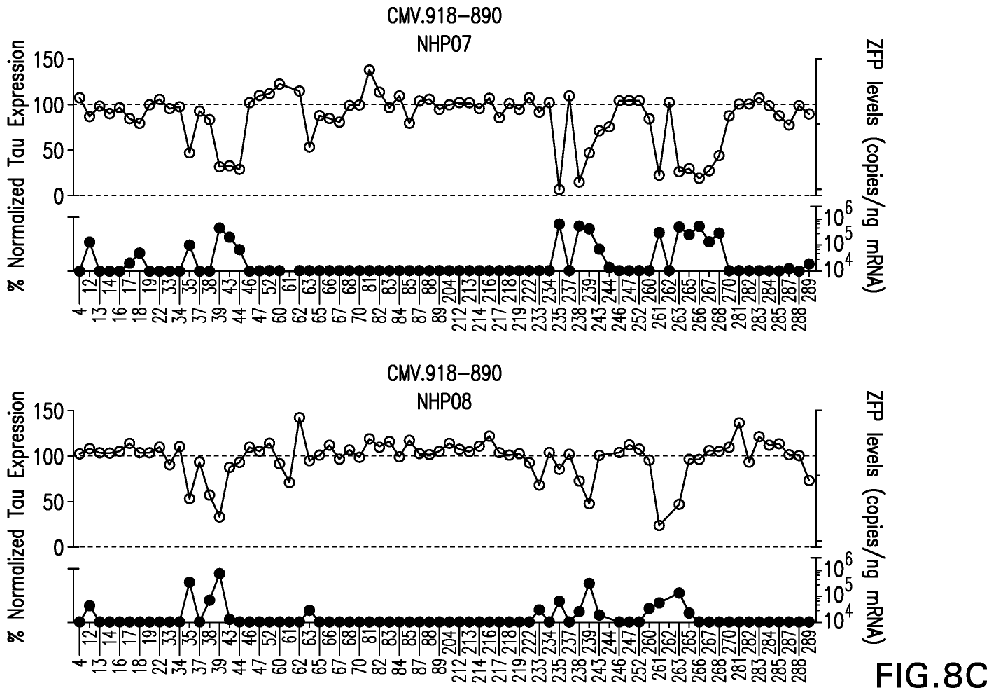

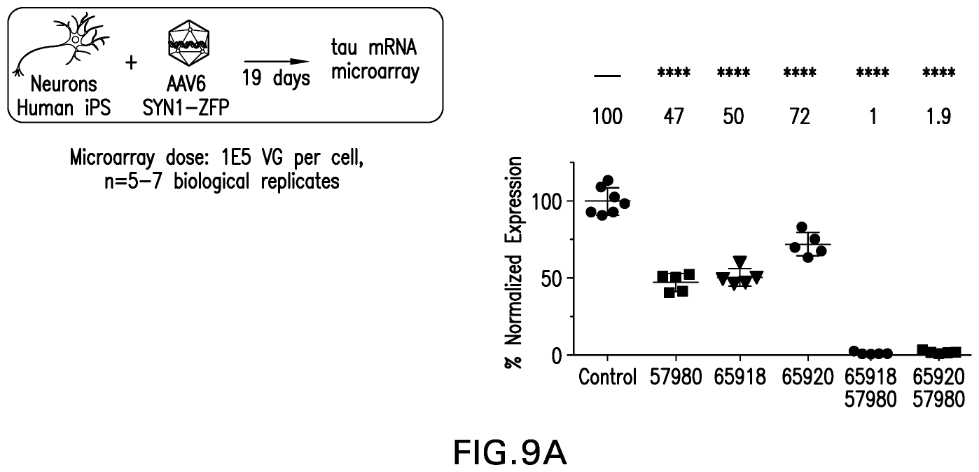

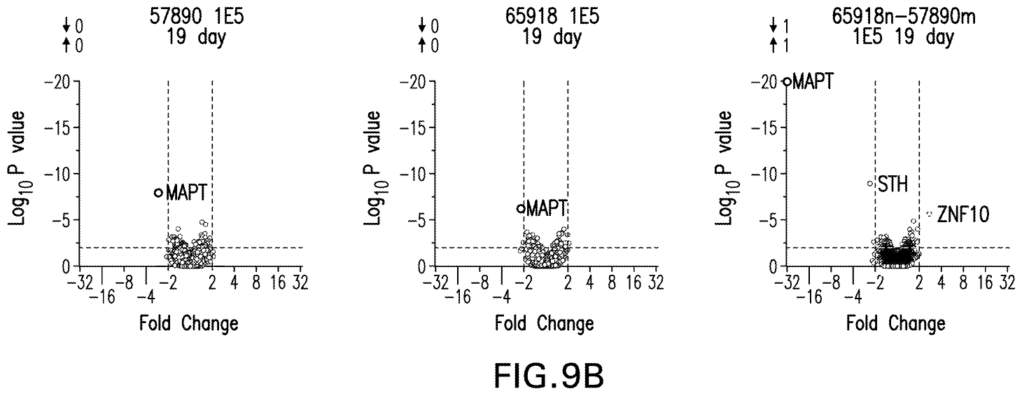

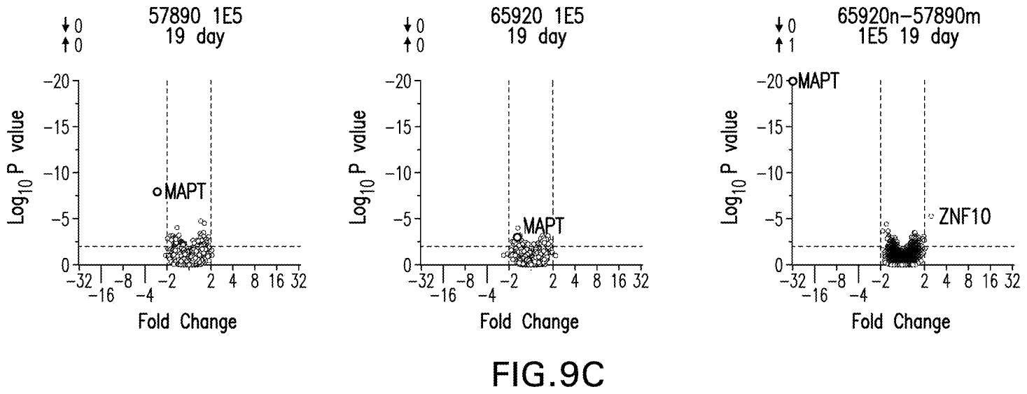

As shown in FIG. 8, tau expression in subjects receiving genetic modulators comprising at least two artificial transcription factors as described herein was significantly repressed as compared to control (vehicle) subjects.

The studies demonstrate that the genetic modulators of the invention modulate gene expression (including at therapeutic levels) in vivo in a primate brain.

The data demonstrate that the genetic repressors described herein are highly active, with spacing independent (up to about 600 bp between target sites and up to about 300 or more base pairs from TSS) saturating repression achieved across up to 3.5 logs of ZFP dose levels. Moreover, the genetic repressors are highly specific in that few to no off-targets were identified. Finally, genetic repressors can be delivered in mRNA form or using a viral vector (e.g., AAV such as AAV9) and show high activity and specificity in vitro and in vivo.

### Example 5: ZFP-TF Activity in Human iPS Neurons

AAV2/6 was used to infect human iPS-derived neurons at about 1E5 VG/cell (iCell Neurons, Cellular Dynamics International Inc). After about 19 days, total RNA was extracted and expression of human MAPT, ZFP-KRAB, and three reference genes (ATP5b, EIF4a2, GAPDH) was assessed using real-time RT-qPCR.

ZFP-TF specificity was also assessed in human iPS-derived neurons at 1E5 VG/cell (iCell Neurons, Cellular Dynamics International Inc). 5-7 biological replicates of each treatment were used, consisting of about 1e5 VG/cell of AAV6 ZFP-TF. After about 19 days post infection, total RNA was extracted and processed via the manufacturer's protocol (Affymetrix Human Clariom S Pico). Robust Multi-array Average (RMA) was used to normalize raw signals from each probe set. Analysis was performed using Transcriptome Analysis Console 4.0 (Affymetrix) with the “Gene Level Differential Expression Analysis” option. ZFP-transfected samples were compared to samples that had been treated with an irrelevant ZFP-TF (that does not bind to MAPT target site). Change calls are reported for transcripts (probe sets) with a >2 fold difference in mean signal relative to control, and a FDR P-value <0.05 (one-way ANOVA analysis, unpaired T-test for each probeset).

The results demonstrated that the specific ZFP-TF combinations displayed synergistic activity in repressing the expression of MAPT in human cells.

All patents, patent applications and publications mentioned herein are hereby incorporated by reference for all purposes in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A composition comprising two or more artificial transcription factors, each artificial transcription factor comprises a DNA-binding domain and functional domain, wherein the artificial transcription factors synergistically modulate gene expression in a cell.

2. The composition of claim 1, wherein the cell is isolated or is in a living subject.

3. The composition of claim 1, wherein the synergistic modulation is at least 2 fold as compared to individual transcription factors.

4. The composition of claim 1, wherein the DNA-binding domain binds to a target site of 12 or more nucleotides.

5. The composition of claim 1, wherein the DNA-binding domain of each transcription factor comprises a zinc finger protein (ZFP), TAL-effector domain, and/or a sgRNA of CRISPR/Cas system.

6. The composition of claim 1, wherein the functional domain comprises a transcriptional activation domain, a transcriptional repression domain, a domain from a DNMT protein such as DNMT1, DNMT3A, DNMT3B, DNMT3L, a histone deacetylase (HDAC), a histone acetyltransferase (HAT), a histone methylase, or an enzyme that sumolyates or biotinylates a histone and/or other enzyme domain that allows post-translation histone modification regulated gene repression.

7. The composition of claim 1, wherein the two or more artificial transcription factors:
(i) bind to any target site of at least 12 nucleotides in a selected target gene;
(ii) bind to target sites within 10,000 or more base pairs of each other;
(iii) bind to target sites within 0 to 300 base pairs on either side of the transcription start site (TSS) of the target gene to be modulated; and/or
(iv) bind to the sense and/or anti-sense strand in a double stranded target.

8. The composition of claim 7, wherein the target gene is a tau (MAPT) gene, a Htt gene, a mutant Htt gene, a mutant C9orf72 gene, a SNCA gene, a SMA gene, an ATXN2 gene, an ATXN3 gene, a PRP gene, an Ube3a-ATS encoding gene, a DUX4 gene, an PGRN gene, a MECP2 gene, an FMR1 gene, a CDKL5 gene, or a LRKK2 gene.

9. The composition of claim 1, wherein the two or more artificial transcription factors are gene repressors.

10. The composition of claim 9, wherein the repressors repress expression of the target gene by at least 50% to 100% as compared to wild-type expression levels.

11. The composition of claim 1, wherein the activity of the functional domain is regulated by an exogenous small molecule or ligand such that interaction with the cell's transcription machinery will not take place in the absence of the exogenous ligand.

12. A pharmaceutical composition comprising the composition of claim 1.

13. A method of modulating gene expression in a subject with a central nervous system (CNS) disease or disorder, the method comprising:
administering a composition according to claim 1 to a subject in need thereof.

14. The method of claim 13, wherein the CNS disease or disorder is Huntington's Disease (HD), Amyotrophic lateral sclerosis (ALS), a prion disease, Parkinson's Disease (PD), dementia with Lewy bodies (DLB) and/or a tauopathy.

15. The method of claim 13, wherein the composition comprising the synergistic artificial transcription factors is provided using one or more polynucleotides.

16. The method of claim 15, wherein the one or more polynucleotides are viral or non-viral vectors.

17. The method of claim 16, wherein the viral vector is an adenovirus vector, a lentiviral vector (LV) and/or adenovirus associated viral vector (AAV).

18. The method of claim 16, wherein the non-viral vector is a plasmid and/or single- or multi-cistronic mRNA.

19. The method of claim 14, the tauopathy is treated by repressing MAPT gene expression; ALS is treated by repressing mutant C9orf72 gene expression; prion disease is treated by repressing prion expression; PD or DLB is treated by repressing α-synuclein expression and/or HD is treated by repressing Htt gene expression.

20. The method of claim 13, wherein gene expression is reduced for a period of 4 weeks, 3 months, 6 months to year or more in the brain of subject.

21. The method of claim 13, wherein the composition is administered to the frontal cortical lobe, the parietal cortical lobe, the occipital cortical lobe; the temporal cortical lobe, the hippocampus, the brain stem, the striatum, the thalamus, the midbrain, the cerebellum and/or to the spinal cord of the subject.

22. The method of claim 13, wherein the composition is administered to the subject via intravenous, intramuscular, intracerebroventricular, intrathecal, intracranial, mucosal, oral, intravenous, orbital and/or intracisternal administration.

23. The method of claim 13, wherein the composition is delivered using
(i) an adeno-associated virus (AAV) vector at 10,000-500,000 vector genome/cell;
(ii) a lentiviral vector at MOI between 250 and 1,000;
(iii) a plasmid vector at 0.01-1,000 ng/100,000 cells; and/or
(iv) mRNA at 0.01-3000 ng/100,000 cells.

24. The method of claim 23, wherein the AAV vector is delivered at a dose of 10,000 to 100,000, or from 100,000 to 250,000, or from 250,000 to 500,000 vector genomes (VG)/cell; at a fixed volume of 1-300 μL to the brain parenchyma at 1E11-1E14 VG/mL and/or at a fixed volume of 0.5-10 mL to the CSF at 1E11-1E14 VG/mL.

25. The method of claim 13, wherein gene expression is reduced in the subject is reduced as compared to controls not receiving the genetic modulators as described herein by at least 30%, or 40%.

26. The method of claim 13, wherein the composition modulates gene expression in a neuron.

27. The method of claim 13, wherein the composition is administered to the subject multiple times.

28. The method of claim 13, wherein the modulation of gene expression reduces biomarkers, pathogenic species and/or symptoms of the CNS disease or disorder.

29. The method of claim 28, wherein neurotoxicity, gliosis, dystrophic neurites, spine loss, excitotoxicity, cortical and hippocampal shrinkage, dendritic tau accumulation, cognitive deficits, motor deficits, dystrophic neurites associated with amyloid β plaques, tau pathogenic species, mHtt aggregates, hyperphosphorylated tau, soluble tau, granular tau, tau aggregation, and/or neurofibrillary tangles (NFTs) are reduced.

30. A cell comprising the composition of claim 1 comprising one or more sequences encoding the one or more artificial transcription factors.

31. The cell of claim 30, wherein the sequences encoding the one or more artificial transcription factors are stably integrated into the genome of the cell.

32. The cell of claim 20, wherein the sequences encoding the one or more artificial transcription factors are maintained episomally in the cell.

33. A kit comprising one or more compositions of claim 1.

34. A method of making a composition comprising synergistic artificial transcription factors of claim 1, the method comprising:
screening individual and combinations of two or more artificial transcription factors targeted to a selected gene for their effect on gene expression; and
identifying synergistic combinations of the artificial ZFP-TFs.

35. The method of claim 34, wherein the two or more artificial transcription factors:
(i) bind to target sites and/or comprise functional domains that are 1-600 base pairs apart;
(ii) bind to target sites that are approximately 1 to 80; 160 to 220; 260 to 400; or 500 to 600 base pairs apart;
(iii) comprise functional domains that are separated from each other by approximately 1 to 80; 260 to 400; or 500 to 600 base pairs apart;
(iv) bind to target sites that are within 400 base pairs on either side of the transcription start site (TSS); and/or
(v) bind to the same antisense (−) or sense (+) strand or to different strands in either orientation).

36. The method of claim 34, wherein the synergistic artificial TFs are at least 2-fold more active than the individual TFs.
